(19) United States
(12) Patent Application Publication
(10) Pub. No.: US 2026/0137707 A1
(43) Pub. Date: May 21, 2026

(54) METHODS OF MODULATING ATXN2 EXPRESSION

(71) Applicant: University of Utah Research Foundation, Salt Lake City, UT (US)

(72) Inventors: Daniel R. Scoles, Salt Lake City, UT (US); Stefan M. Pulst, Salt Lake City, UT (US)

(21) Appl. No.: 18/872,754

(22) PCT Filed: Jun. 6, 2023
(86) PCT No.: PCT/US2023/024618

§ 371 Date: Dec. 6, 2024

Related U.S. Application Data
(60) Provisional application No. 63/349,536, filed on Jun. 6, 2022.

(51) Int. Cl. A61K 31/7048; A61K 31/136; A61K 31/235; A61K 31/24; A61K 31/305; A61K 31/353; A61K 31/395; A61K 31/416; A61K 31/4196; A61K 31/4375; A61K 31/4409; A61K 31/4725; A61K 31/473; A61K 31/4745; A61K 31/496; A61K 31/519; A61K 31/52; A61K 31/5375; A61K 31/5377; A61K 31/5415; A61K 31/706; A61K 31/7072; A61K 31/714

(52) U.S. Cl. CPC A61K 31/7048; A61K 31/136; A61K 31/235; A61K 31/24; A61K 31/305; A61K 31/353; A61K 31/395; A61K 31/416; A61K 31/4196; A61K 31/4375; A61K 31/4409; A61K 31/4725; A61K 31/473; A61K 31/4745; A61K 31/496; A61K 31/519; A61K 31/52; A61K 31/5375; A61K 31/5377; A61K 31/5415; A61K 31/706; A61K 31/7072; A61K 31/714

(57) ABSTRACT

The expression of the ATXN2 gene can be modulated to reduce the amount of ataxin-2 protein produced by a cell. This can be accomplished by administering to the cell an effective amount of an ATXN2 modulating agent. Screening was performed with 428,749 compounds for lowering expression of an ATXN2-luciferase reporter in HEK-293 cells, with a multiplexed cell viability assay to ensure target reduction in the absence of cytotoxicity. Secondary qHTS assays included a CMV-luciferase reporter expressed in HEK-293 cells as well as a biochemical assay using recombinant luciferase to detect inhibitors of the reporter enzyme. A diverse set of compounds were found to reduce ATXN2 expression, including compounds targeting HSP90, cardiac glycosides, NaK-ATPases and topoisomerases.

### CROSS REFERENCE TO RELATED APPLICATIONS

This application claims priority to U.S. Provisional Application No. 63/349,536, filed Jun. 6, 2022, which is incorporated herein by reference.

### STATEMENT REGARDING FEDERALLY SPONSORED RESEARCH OR DEVELOPMENT

This invention was made with government support under NS073009, and NS103883 awarded by the National Institutes of Health. The government has certain rights in the invention.

### NAMES OF THE PARTIES TO A JOINT RESEARCH AGREEMENT

Note applicable.

### INCORPORATION BY REFERENCE STATEMENT

Not applicable.

### FIELD OF THE DISCLOSURE

The present disclosure relates to compounds, compositions and methods of modulating protein expression for therapeutic purposes. Accordingly, the present disclosure relates generally to the fields of biology, cell physiology, chemistry, pharmaceutical sciences, medicine, and other health sciences.

### BACKGROUND OF THE DISCLOSURE

Neurodegenerative diseases occur when nerve cells in the brain or peripheral nervous system lose function over time and ultimately die. Further, nerve cells generally don't reproduce or replace themselves. The risk of being affected by a neurodegenerative disease increases with age. Non-limiting examples of neurodegenerative diseases include peripheral neuropathy, Alzheimer's disease (AD), Parkinson's disease (PD), Huntington's disease (HD), spinocerebellar ataxia (SCA), prion disease, motor neuron disease (MND), amyotrophic lateral sclerosis (ALS), multiple sclerosis (MS), and spinal muscular atrophy (SMA) among others.

### BRIEF DESCRIPTION OF THE DRAWINGS

FIG. 1 is a schematic illustration of an ATXN2-luc gene cassette used in a plasmid pGL2h-5A3.

FIG. 2 is a graph of raw, untransformed data of a pilot screen in which H2 cells were treated with compounds for 24 hrs at a single 10 M dose, then assayed for luciferase.

FIG. 3A is a graph of CMV-luc response and viability vs. dose of ChemBridge 5553825 in HEK-293 cells transfected with CMV-luc.

FIG. 3B is a graph of ATXN2-luc response and viability vs. dose of ChemBridge 5553825 in S2 cells.

FIG. 3C is a graph of ATXN2-Rluc response vs. dose of ChemBridge 5553825 in cells transfected with ATXN2-Rluc.

FIG. 3D is a graph of luciferase units vs. well column number in S2 cells treated with ChemBridge 5553825 at 1 μM or with a vehicle (1% DMSO).

FIG. 3E shows means and standard deviations of luciferase units for the cells treated with vehicle or ChemBridge 5553825.

FIG. 4A shows S2 cells plated at 1,000 cells/well.

FIG. 4B shows the effect of increasing S2 cell abundance on luminescence and fluorescence signals.

FIG. 4C shows a preliminary miniaturized compound screen using S2 with compound diluent alone (DMSO) or 5 doses of LOPAC library compound (Sigma).

FIG. 5 shows Z′-score vs. plate number for a steady-Glo primary screen and CellTiter Fluor viability screen using the ATXN2-luc S2 cells and 363,021 library compounds.

FIG. 6A shows graphs of relative luciferase units for cells expressing ATXN2-luc and CMV-luc and a graph of viability when the cells are exposed to ganetespib.

FIG. 6B shows graphs of relative luciferase units for cells expressing ATXN2-luc and CMV-luc and a graph of viability when the cells are exposed to NVP-AUY922.

FIG. 6C shows graphs of relative luciferase units for cells expressing ATXN2-luc and CMV-luc and a graph of viability when the cells are exposed to SNX-5422.

FIG. 6D shows graphs of relative luciferase units for cells expressing ATXN2-luc and CMV-luc and a graph of viability when the cells are exposed to SNX-2112.

FIG. 6E shows graphs of relative luciferase units for cells expressing ATXN2-luc and CMV-luc and a graph of viability when the cells are exposed to AT-13387AU.

FIG. 6F shows graphs of relative luciferase units for cells expressing ATXN2-luc and CMV-luc and a graph of viability when the cells are exposed to CNF-2024.

FIG. 6G shows graphs of relative luciferase units for cells expressing ATXN2-luc and CMV-luc and a graph of viability when the cells are exposed to tanespimycin.

FIG. 6H shows graphs of relative luciferase units for cells expressing ATXN2-luc and CMV-luc and a graph of viability when the cells are exposed to retaspimycin.

FIG. 6I shows graphs of relative luciferase units for cells expressing ATXN2-luc and CMV-luc and a graph of viability when the cells are exposed to KW-2478.

FIG. 6J shows graphs of relative luciferase units for cells expressing ATXN2-luc and CMV-luc and a graph of viability when the cells are exposed to VER-82576.

FIG. 6K shows graphs of relative luciferase units for cells expressing ATXN2-luc and CMV-luc and a graph of viability when the cells are exposed to proscillaridin A.

FIG. 6L shows graphs of relative luciferase units for cells expressing ATXN2-luc and CMV-luc and a graph of viability when the cells are exposed to ouabain.

FIG. 6M shows graphs of relative luciferase units for cells expressing ATXN2-luc and CMV-luc and a graph of viability when the cells are exposed to digoxin.

FIG. 7A is a graph of ATXN2-luc response vs. dose of proscillaridin A in H2 cells.

FIG. 7B is a graph of ATXN2 production in HEK-293 cells vs. dose of proscillaridin A.

FIG. 7C is a graph of ATXN2-luc expression (top) of S2 cells that were transfected with ATP1A2, and the cell viability (bottom) determined by CellTiter-Fluor assay.

FIG. 7D is a graph of ATXN2 expression means and standard deviations from independent transfections, including HEK-293 cells transfected with ATP1A2, each analyzed by qPCR.

FIG. 7E is a graph of ATP1A2 and ATXN2 transcription means and standard deviations in HEK-293 cells with reduced expression of ATP1A2 by RNA interference.

FIG. 8 shows luciferase mRNA vs. concentration of 17-DMAG when treating H2 cells.

FIG. 9A shows ATXN2-luc response and viability vs. concentration of 17-DMAG used to treat H2 cells.

FIG. 9B shows the mean and standard deviation of expression of endogenous ATXN2 in HEK-293 cells at various concentrations of 17-DMAG.

FIG. 9C shows a reduction of endogenous non-mutant ATXN2 protein with increasing concentration of 17-DMAG determined by western blotting.

FIG. 9D shows a reduction of ATXN2 protein with increasing concentration of 17-DMAG.

FIG. 9E shows an increase in HSP70 with increasing concentration of 17-DMAG, which indicates inhibition of HSP90.

FIG. 10A shows several proteins in non-mutated cells (Q22) and mutated cells (ATXN2-Q58) at multiple concentrations of 17-DMAG.

FIG. 10B shows means and standard deviations of these proteins in the non-mutated cells and mutated cells at multiple concentrations of 17-DMAG.

FIG. 11A shows ATXN2-Q22 and HSP70 proteins for mice treated with 17-DMAG, with the mouse ID numbers above the lanes.

FIG. 11B shows the means and standard deviations of ATXN2-Q22 in mice treated with the vehicle and with 17-DMAG.

FIG. 11C shows the means and standard deviations of HSP70 in mice treated with the vehicle and with 17-DMAG.

FIG. 12A shows ATXN2-Q22 and HSP70 proteins for mice treated with HSP990, with the mouse ID numbers above the lanes.

FIG. 12B shows the means and standard deviations of ATXN2-Q22 in mice treated with the vehicle and with HSP990.

FIG. 12C shows the means and standard deviations of HSP70 in mice treated with the vehicle and with HSP990.

It should be noted that the figures are merely exemplary of several embodiments and no limitations on the scope of the present invention are intended thereby. Further, the figures are generally not drawn to scale, but are drafted for purposes of convenience and clarity in illustrating various aspects of the invention.

### DESCRIPTION OF EMBODIMENTS

While exemplary embodiments are described in sufficient detail to enable those skilled in the art to practice the disclosure, it should be understood that other embodiments may be realized and that various changes to the disclosure may be made without departing from the spirit and scope of the present disclosure. Thus, the following more detailed description of the embodiments of the present disclosure is not intended to limit the scope of the disclosure, as claimed, but is presented for purposes of illustration only and not limitation to describe the features and characteristics of the present disclosure, to set forth the best mode of operation of the disclosure, and to sufficiently enable one skilled in the art to practice the disclosure. Accordingly, the scope of the present disclosure is to be defined solely by the appended claims.

### Definitions

In describing and claiming the present disclosure, the following terminology will be used.

The singular forms “a,” “an,” and “the” include plural referents unless the context clearly dictates otherwise. Thus, for example, reference to “a peptide” includes reference to one or more of such peptides and reference to “the compound” refers to one or more of such compounds.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility and imprecision associated with a given term, metric or value. Unless otherwise stated, use of the term “about” in accordance with a specific number or numerical range should also be understood to provide support for such numerical terms or range without the term “about”. For example, for the sake of convenience and brevity, a numerical range of “about 50 angstroms to about 80 angstroms” should also be understood to provide support for the range of “50 angstroms to 80 angstroms.” Furthermore, it is to be understood that in this written description support for actual numerical values is provided even when the term “about” is used therewith. For example, the recitation of “about” 30 should be construed as not only providing support for values a little above and a little below 30, but also for the actual numerical value of 30 as well. The degree of flexibility for a particular variable can be readily determined by one skilled in the art. However, unless otherwise enunciated, the term “about” generally connotes flexibility of less than 2%, and most often less than 1%, and in some cases less than 0.01%.

As used herein, the terms “therapeutic agent,” “active agent,” and the like can be used interchangeably and refer to agent that can have a beneficial or positive effect on a subject when administered to the subject in an appropriate or effective amount.

As used herein, “modulates,” “modulating,” and the like refer to an alteration of an amount of an agent or an activity or process as compared to the native or natural state thereof. For example, “modulating” expression of a protein refers to acting upon the native expression process in a way that either increases (e.g. upregulates) or decreases (e.g. downregulates) expression. Further, modulating a substance or agent refers to increasing or decreasing the concentration or amount of substance. For example, modulating ataxin-2 protein refers to increasing or decreasing an amount or concentration of ataxin-2 protein, for example, in a cell. Moreover, “modulating” ATXN2 expression can refer to either increasing or decreasing expression of the ATXN2 gene.

As used herein, the terms “inhibit,” “inhibiting,” “inhibition,” or the like are used to refer to a variety of inhibition degrees and techniques. For example, such terms can refer to at least a reduction of a substance or occurrence of an event (e.g. expression of a gene) and also encompasses complete absence of a substance or cessation of the event. When modified by terms such as “complete” or “total” or other like verbiage, the use of “inhibit” and like terms refers to complete cessation or arrest of the production of an agent or expression of a gene. It should be understood that as used in this written description, the use of“inhibit,” “inhibiting,” or the like includes express support for the use of such term together with the above-recited modifiers as if present. As such, instances of “inhibit” or “inhibiting,” and the like include express support for “completely inhibits,” total inhibition,” “fully inhibit,” “absolutely inhibiting,” etc.

In addition, “inhibit,” “inhibiting,” or the like refer to any process or mechanism by which inhibition can be achieved or applied for an identified substance or event. For example, gene expression can be “inhibited” by pre- and/or post-transcriptional inhibition. With respect to pre-transcription inhibition, “inhibit” or “inhibiting” can refer to preventing or reducing transcription of a gene, inducing altered transcription of a gene, and/or reducing a rate of transcription of a gene, whether permanent, semi-permanent, or transient. Thus, in some examples, “inhibit” or “inhibiting” can refer to permanent changes to the DNA, whereas in other examples no permanent change to the DNA is made. With respect to post-transcriptional inhibition, “inhibit” or “inhibiting” can refer to preventing or reducing translation of a genetic sequence to a protein, inducing an altered translation of a genetic sequence to an altered protein (e.g. as misfolded protein, etc.), and/or reducing a rate of translation of a genetic sequence to a protein, whether permanent, semi-permanent, or transient. In some specific examples, “inhibit” or “inhibiting” can refer to pre-transcriptional inhibition. In other specific examples, “inhibit” or “inhibiting” can refer to post-transcriptional inhibition. Of course, the type of inhibition can depend on the specific type(s) of inhibitor(s) or therapeutic agent(s) employed. Thus, “inhibit” or “inhibiting” can include any decrease in expression of a gene as compared to native expression, whether pre- or post-transcriptional, partial or complete.

As used herein, the terms “formulation” and “composition” are used interchangeably and refer to a mixture of two or more compounds, elements, or molecules. In some aspects, the terms “formulation” and “composition” may be used to refer to a mixture of one or more active agents with a carrier or other excipients.

As used herein, a “subject” refers to a mammal that may benefit from the method or device disclosed herein. Examples of subjects include humans, and may also include other animals such as horses, pigs, cattle, dogs, cats, rabbits, and aquatic mammals. In one specific aspect, the subject is a human.

As used herein, an “effective amount” or a “therapeutically effective amount” of a drug refers to a non-toxic, but sufficient amount of the drug, to achieve a desired effect, such as a therapeutic effect. It is understood that in biological tissues and systems, various biological factors may affect the ability of a substance or agent to perform its intended task. Therefore, an “effective amount” or a “therapeutically effective amount” may be dependent in some instances on such biological factors. Further, while the achievement of therapeutic effects may be measured by a physician or other qualified medical or technically-trained research personnel using evaluations known in the art, it is recognized that individual variation and response to processes, such as treatments may make the achievement of therapeutic effects a somewhat subjective decision. The determination of an effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986), incorporated herein by reference. In the specific context of the ATXN2 modulating agents described herein, an effective amount can include an amount sufficient to modulate, for example reduce, expression of ATXN2 as compared to expression of ATXN2 by a cell to which an ATXN2 modulating agent is not or has not been administered.

In this disclosure, “comprises,” “comprising,” “containing” and “having” and the like can have the meaning ascribed to them in U.S. patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. patent law. “Consisting essentially of” or “consists essentially of” have the meaning generally ascribed to them by U.S. patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open-ended term, like “comprising” or “including,” in the written description it is understood that direct support should be afforded also to “consisting essentially of” language as well as “consisting of” language as if stated explicitly and vice versa.

This written description may describe classes, genera, or other groups of individuals or specific elements, compounds, molecules, agents, nucleotides, or species which are presented together for some reason of commonality or convenience, for example, a common structure, activity, characteristic, property, behavior, etc. For example, several groups of chemical compounds or chemical species are presented and/or described below. It is to be understood that any group of individuals (e.g. elements, compounds, molecules, etc.) articulated in this written description provides express support for both the identified group and each of its identified species. For example, if reference is made to a group using a genus name or terminology, such recitation provides express support for each individual member or species of the group, as well as for subsets or subgroups containing or excluding select members or species of the group. Further, recitation of a member or species of a group provides express support for the genus of the group and also for other individual members of the group, including subsets and subgroups. The foregoing principle and practice extends to both the inclusion and exclusion of recited species within a genus. For example, in this written description, the mere mention or recitation of a group by its genus name or other characterization, or of one or more individual species or subgroup of species therein, provides support for both its presence (i.e. inclusion) or absence (i.e. exclusion) with one or more other groups or members of a group. For example, a recitation of “Group A, Group B, and Group C,” provides support for the inclusion of such groups together, or the exclusion of one or more of the group members. As such, express support is afforded for all variations of the grouping, namely, “Group A, Group B, and Group C,” as well as “Group A and Group B, but not Group C,” “Group A and Group C, but excluding Group B,” “Group B and Group C, but excluding Group A,” as well as just “Group A,” “Group B,” or “Group C” individually. Likewise, if Group A or Genus A is recited to include members or species 1-10, such identification or recitation is understood to provide express support for each species individually, as well as any and all possible subgroups of species as well as the affirmative exclusion of any of the recited species. For example, a recitation or identification of Group or Genus A including species 1-10, provides express support for the subgroup of Species 2-6, individual Species 1, individual Species 9, and subgroup Species 1-9, both in an affirmative and exclusionary context, for example, Genus A excluding Species 1, Genus A excluding Species 2-8, Genus A excluding Species 6, etc.

As used herein, comparative terms such as “increased,” “decreased,” “better,” “worse,” “higher,” “lower,” “enhanced,” “improved,” “maximized,” “minimized,” and the like refer to a property of a device, component, composition, biologic response, biologic status, or activity that is measurably different from other devices, components, compositions, biologic responses, biologic status, or activities that are in a surrounding or adjacent area, that are similarly situated, that are in a single device or composition or in multiple comparable devices or compositions, that are in a group or class, that are in multiple groups or classes, or as compared to an original (e.g. untreated) or baseline state, or the known state of the art. For example, “improved” symptoms of a neurodegenerative disease can refer to symptoms that are less severe compared to untreated, baseline symptoms in a subject.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a defacto equivalent of any other member of the same list solely based on their presentation in a common group without indications to the contrary.

As used herein, the term “at least one of” is intended to be synonymous with “one or more of.” For example, “at least one of A, B and C” explicitly includes only A, only B, only C, or combinations of each.

Concentrations, amounts, levels and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges or decimal units encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

Occurrences of the phrase “in one embodiment,” or “in one aspect,” herein do not necessarily all refer to the same embodiment or aspect. Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

### EXAMPLE EMBODIMENTS

An initial overview of disclosure embodiments is provided below and specific embodiments are then described in further detail. This initial summary is intended to aid readers in understanding the technological concepts more quickly, but is not intended to identify key or essential features thereof, nor is it intended to limit the scope of the claimed subject matter.

Spinocerebellar ataxia type 2 (SCA2) is a debilitating neurodegenerative disorder characterized primarily by gait ataxia, for which there are no disease-modifying treatments. SCA2 is caused by a CAG repeat expansion mutation in the ATXN2 gene in an encoded region that results in polyglutamine (polyQ) expansion of the ATXN2 protein. In normal individuals ATXN2 usually contains 22 CAGs interrupted by one or two CAA codons, while SCA2 occurs when ATXN2 contains pure CAG tracts numbering 33 or more. SCA2 is also characterized by anticipation where in families CAG repeat lengths generally increase with each generation, and increased repeat lengths are associated with earlier age of onset and greater disease severity. SCA2 is included among nine total polyQ diseases including DRPLA, SMBA, HD, and SCAs 1-3, 6, 7, & 17, each of which are characterized phenotypically by progressive neurodegeneration. Ataxia in SCA2 results from progressive loss of cerebellar Purkinje cells as for most of the nearly 40 SCAs, and pathological defects in the brain stem are observed, mainly involving the pontine and olivary nuclei.

Since the discovery of ATXN2 as the SCA2 gene in 1996, efforts have been made to characterize ATXN2 function with the intention to identify therapeutic targets for SCA2. SCA2 is characterized by toxic gain of function mutations in the ATXN2 gene. A principal ATXN2 function is regulation of RNA processing. Evidence for this includes that the majority of known ATXN2 interacting proteins are RNA binding proteins (RBPs), including A2BP1/Fox1, DDX6, PABP1, TDP-43, and FUS. ATXN2 also localizes to p-bodies and stress granules supporting a role as a regulator of RNA translation or stability. Consistent with this, transcriptome profiling of cerebellar tissues from Atxn2 knockout mice identified several upregulated ribosomal proteins and abnormal translation. ATXN2 interacts with polyribosomes, and Atxn2 loss altered activation of 4E-BP1 and S6 via mTOR. ATXN2 also interacts with the RNA binding protein Staufen 1 (STAU1) and both are localized to stress granules. STAU1 and mTOR are both overabundant in SCA2 and ALS patient fibroblasts and mouse models associated with abnormal autophagy which can be rescued by RNAi targeting STAU1, ATXN2 or MTOR.

Transcriptomic profiling of cerebellar tissues from SCA2 BAC-Q72 mice also showed the presence of severely reduced Rgs8 mRNA levels, as well as impaired Rgs8 translation related to an abnormal interaction between polyQ expanded ATXN2 and the Rgs8 mRNA. ATXN2 mutation is also associated with abnormal calcium homeostasis, and SCA2 Purkinje cells are characterized by elevated cytoplasmic Ca2+. This is caused in part by abnormal interaction by polyQ expanded ATXN2 with InsP3R resulting in increased Ca2+ release from the endoplasmic reticulum (ER). However, elevated cytoplasmic Ca2+ may also result from impaired Rgs8 expression since RGS proteins may inhibit mGlur1, which is a positive regulator of Ca2+ release from internal stores. Mutant ATXN2 also interacts with endophilin A1 and endophilin A3 and was found in an endophilin complex with CIN85, indicating that ATXN2 has a role in endophilin-CIN85-Cbl mediated endocytosis.

It remains unclear which of the pathways regulated by mutant ATXN2 drive SCA2 pathogenesis. However, the restoration of autophagy by ATXN2 siRNA indicates lowering its expression would be therapeutic, and this is also supported by improved motor and molecular and neurophysiological phenotypes in SCA2 mice treated by an antisense oligonucleotide (ASO) targeting ATXN2. In the present disclosure, small molecules are disclosed that can lower overall ATXN2 expression toward developing a therapeutic for SCA2. Proof of principal data supporting this approach exists for other polyglutamine diseases. Additionally, it has been demonstrated that knockout of the Atxn2 gene in mice resulted in no neurodegenerative phenotype. The present disclosure describes a quantitative high throughput screen (qHTS) for compounds lowering ATXN2 expression. The screening identified multiple major classes of compounds that lower ATXN2 expression that may include lead compounds that could be modified by medicinal chemistry toward production of SCA2 therapeutics. Several hit compounds, including HSP90 inhibitors and cardiac glycosides, were characterized in additional ATXN2 assays and are the main focus of this disclosure.

The compounds described herein that can reduce ATXN2 expression are referred to as ATXN2 modulating agents. In some examples, methods of modulating ATXN2 expression in a cell can include administering to the cell an effective amount of an ATXN2 modulating agent. In certain examples, the ATXN2 modulating agent can include a compound identified herein. In further examples, the ATXN2 modulating agent can include a cardiac glycoside, an HSP90 inhibitor, an NaK-ATPase inhibitor, a topoisomerase inhibitor, or a combination thereof.

When an ATXN2 modulating agent is administered to a cell, the ATXN2 expression in the cell can be reduced. This can have the effect of reducing a cellular concentration of ataxin-2 protein, which is the protein encoded by the ATXN2 gene. In some examples, a method of reducing a concentration of an ataxin-2 protein in a cell can include administering to the cell an effective amount of an ATXN2 modulating agent. The ATXN2 modulating agent can be any of the compounds described herein.

As explained above, certain diseases can be caused by or related to mutations of the ATXN2 gene. As used herein, the term “ATXN2” can refer to the normal ATXN2 and to the mutated ATXN2 gene. Similarly, the term “ATXN2 modulating agent” can refer to compounds that reduce the expression of the normal ATXN2 gene, or the mutated ATXN2 gene, or both. It is further noted that the ATXN2 gene can mutate in a variety of different ways. Any form of mutated ATXN2 gene can be referred to using the term “ATXN2.” In certain examples, the ATXN2 gene in question can be mutated in a way that causes SCA2. In particular examples, the ATXN2 gene can be mutated such that the gene includes a tract of repeating CAG codons having 33 or more consecutive CAG codons. Many ATXN2 modulating agents can effectively reduce the expression of both normal ATXN2 genes and mutated ATXN2 genes. In some cases, the “normal” ATXN2 gene can be referred to as a “wild-type” ATXN2 gene.

The ataxin-2 protein that is produced from the ATXN2 gene can also vary in structure when the ATXN2 gene is mutated. The term “ataxin-2” as used herein can refer to the normal ataxin-2 protein and to the mutated protein. In certain examples, the ataxin-2 protein can include a polyglutamine expansion caused by a tract of repeated CAG codons in a mutated ATXN2 gene.

A variety of specific ATXN2 modulating agents are identified below. These agents can be categorized within several groups depending on known functions of the compounds. In some examples, the ATXN2 modulating agent can be a cardiac glycoside, an HSP90 inhibitor, an NaK-ATPase inhibitor, a topoisomerase inhibitor, or a combination thereof. Other ATXN2 modulating agents can also be used that may not fall within these categories. In further examples, the ATXN2 modulating agent can be any compound or combination of compounds identified in Table 1, Table 3S, or Table 4S below.

In certain examples, the ATXN2 modulating agent can be a cardiac glycoside. Cardiac glycosides are a class of organic compounds that increase the output force of the heart and decrease its rate of contractions. In some examples, cardiac glycosides can have a molecular structure including a steroid nucleus attached to a sugar (glycoside) group and an R group. Other functional groups can be attached to the steroid nucleus, such as methyl, hydroxyl, and aldehyde groups. The glycoside group and the R group can also vary between different cardiac glycosides. Non-limiting examples of cardiac glycosides that can be used at ATXN2 modulating agents include strophantin octahydrate, dihydroouabain, lanatoside B, helveticoside, ouabain, and combinations thereof. In certain examples, the ATXN2 modulating agent can be a cardiac glycoside excluding proscillaridin A, digoxin, digitoxigenin, and strophanthidin.

In further examples, the ATXN2 modulating agent can be an NaK-ATPase inhibitor. This is a class of compounds that overlaps with cardiac glycosides, and many compounds can be both cardiac glycosides and NaK-ATPase inhibitors. These compounds inhibit the cellular sodium-potassium ATPase pump. This can increase the output force of the heart and decrease the rate of heat contractions. Non-limiting examples of ATXN2 modulating agents that are NaK-ATPase inhibitors include strophantin octahydrate, dihydroouabain, lanatoside B, helveticoside, ouabain, and combinations thereof. In certain examples, the ATXN2 modulating agent can be an NaK-ATPase inhibitor excluding proscillaridin A, digoxin, digitoxigenin, and strophanthidin.

The ATXN2 modulating agent can also be an HSP90 inhibitor. These compounds can inhibit the activity of the HSP90 heat shock protein. Non-limiting examples of HSP90 inhibitors that can be used as ATXN2 modulating agents include HSP990, Ganetespib, Luminespib, SNX-5422, SNX-2112, Onalespib, CNF-2024, Tanespimycin, Retaspimycin, KW-2478, VER-82576, and combinations thereof. In certain examples, the ATXN2 modulating agent can be an HSP90 inhibitor excluding 17-DMAG (alvespimycin).

In other examples, the ATXN2 modulating agent can be a topoisomerase inhibitor. Topoisomerase inhibitors are compounds that block the ligation step of the cell cycle, which generates DNA single and double-strand breaks, leading to apoptotic cell death. Blocking DNA generation can prevent cell splitting and growth. Non-limiting examples of topoisomerase inhibitors that can be used as ATXN2 modulating agents include camptothecin, vosaroxin, amonafide, etoposide, mitoxantrone dihydrochloride, and combinations thereof.

The ATXN2 modulating agents described herein can have varying effectiveness for reducing ATXN2 expression, and thereby reducing a cellular concentration of ataxin-2 protein. The effectiveness of the agents can be characterized in multiple different ways. In some examples, the ATXN2 modulating agent can reduce ATXN2 expression by greater than about 50%. In further examples, the ATXN2 modulating agent can reduce ATXN2 expression by greater than about 60%, or 70%, or 80%, or 90%. As a result, the ATXN2 modulating agent can reduce ataxin-2 concentration in the cell by greater than 50%, or 60%, or 70%, or 80%, or 90%. These reductions can be with respect to a cell that has not be exposed to the ATXN2 modulating agent. The reduction of ATXN2 expression can be achieved by exposing the cell to a dose of ATXN2 modulating agent from about 10 nanomolar (nM) to about 10 micromolar (μM) in some examples. In other examples, the does can be from 10 nM to 100 nM, or from 10 nM to 500 nM, or from 10 nM to 1 μM, or from 500 nM to 1 μM, or from 1 μM to 10 μM.

The effectiveness of the ATXN2 modulating agent can also be characterized by its half maximal inhibitor concentration (IC50), which is the concentration at which the ATXN2 modulating agent has reduced the ATXN2 expression by half as much as the maximum inhibitory effect of the ATXN2 at any higher concentration. In some examples, the ATXN2 modulating agent can have an IC50 from about 17 nM to about 57 μM. In further examples, the IC50 can be from about 20 nM to about 20 μM, or from about 20 nM to about 1 μM, or from about 20 nM to about 500 nM, or from about 20 nM to about 100 nM.

The cells treated with the ATXN2 modulating agent can also have a suitably high cell viability. Cells treated with the ATXN2 modulating agent at the doses described above can have a viability greater than about 50%, or greater than about 65%, or greater than about 80%, or greater than about 90%, in some examples.

The effects of the ATXN2 modulating agent can be applied to cells in vivo, in vitro, or both. As used herein, “in vivo” refers to effects of an ATXN2 modulating agent in a living organism such as a mammal, and in some cases in a human subject. As used herein, “in vitro” refers to the effects of an ATXN2 modulating agent on cells in a test tube, culture dish, well plate, or other location that is not in a living organism. It is noted that the cells can be alive in an in vitro use of the ATXN2 modulating agent, but the cells are not part of a larger living organism such as a mammal or human subject.

The screening assays described below involved testing a large number of chemical compounds to see which compounds would reduce ATXN2 expression in cells. Varying levels of ATXN2 expression were observed after treating cells with all these compounds. The most effective compounds were identified as the compounds that reduced the ATXN2 expression by several standard deviations below the mean of the entire group of assayed compounds. In some examples, the ATXN2 modulating agents can include compounds from the assays that reduced ATXN2 to a level more than 2.7 standard deviations below the mean, or more than 3 standard deviations below the mean, or more than 4 standard deviations below the mean, or more than 5 standard deviations below the mean. Table 1, below, shows many compounds that reduced ATXN2 expression by greater than 2.7 standard deviations below the mean. In some examples, the methods described herein can utilize ATXN2 modulating agents from Table 1. Tables 3S and 4S also list compounds that can be effective ATXN2 modulating agents. In some examples, the methods described herein can utilize ATXN2 modulating agents from Table 1, Table 3S, Table 4S, or a combination thereof. In certain examples, the ATXN2 modulating agent can be a compound or combination of compounds from Table 3S. In other examples, the ATXN2 modulating agent can be a compound or combination of compounds from Table 4S. In still further examples, the ATXN2 modulating agent can be a compound or combination of compounds from Table 3S, Table 4S, or a combination thereof. In certain examples, the ATXN2 modulating agent can be a compound from these tables, but excluding proscillaridin A, digoxin, digitoxigenin, strophanthidin, and 17-DMAG.

In some examples, the ATXN2 modulating agent can be Proscillaridin A; ASN5914674; ChemBridge ID #5156626; ChemBridge ID #5352460; ASN5544487; ASN6088278; ASN6291966; ASN4887530; ASN6353460; RESORCINOL MONOACETATE; ASN6414283; ChemBridge ID #5344759; ChemBridge ID #5228409; ChemBridge ID #5268955; ChemBridge ID #5344191; TRIFLUOPERAZINE HYDROCHLORIDE; ASN5544420; TMB-8; THIMEROSAL; ASN5819045; ASN6353027; ASN7185473; AST 6978215; ChemBridge ID #5195242; ASN6087944; Camptothecine (S+); ASN6956162; ASN6353461; ASN4886968; ASN5544494; Trifluridine; ChemBridge ID #5367872; COBALAMINE; ChemBridge ID #5427770; ChemBridge ID #5349968; ASN5114176; ASN6353563; TOMATINE; ASN5262651; ASN4420965; U-37883A; SU1498; PINACIDIL; ChemBridge ID #5223563; ASN6353483; GAMBOGIC ACID; ASN2053976; Emetine dihydrochloride; ChemBridge ID #5354001; ChemBridge ID #5624085; ChemBridge ID #5247688; ChemBridge ID #5378363; Puromycin dihydrochloride; ChemBridge ID #6625505; ASN6088274; ChemBridge ID #5306365; ChemBridge ID #5100075; ChemBridge ID #5425925; ChemBridge ID #5510292; ASN5544434; ChemBridge ID #5545700; ChemBridge ID #5378047; ASN6118229; ChemBridge ID #5510261; STROPHANTHIDIN; ChemBridge ID #5936763; ChemBridge ID #5686303; Niclosamide; ChemBridge ID #5341861; ChemBridge ID #5356272; ASN5114173; ASN3777991; ChemBridge ID #5823090; ChemBridge ID #5453149; MITOXANTHRONE HYDROCHLORIDE; LANATOSIDE C; ASN6957234; ChemBridge ID #5411695; GITOXIGENIN DIACETATE; ChemBridge ID #6538857; ChemBridge ID #5353349; ChemBridge ID #5355005; ASN5114006; Daunorubicin hydrochloride; ACRIFLAVINIUM HYDROCHLORIDE; ChemBridge ID #5553825; ChemBridge ID #5617332; ChemBridge ID #5325791; AMINACRINE; DIGITOXIN; CYMARIN; GITOXIN; ASN3778057; ISOPROPAMIDE IODIDE; ChemBridge ID #5238220; ChemBridge ID #5526179; ASN5021309; Cephaeline dihydrochloride heptahydrate; ChemBridge ID #5276688; PERUVOSIDE; AST6018126; Anisomycin; ChemBridge ID #5268879; ChemBridge ID #5930487; ChemBridge ID #5175328; ChemBridge ID #5244378; ChemBridge ID #5105504; ASN3574739; ChemBridge ID #5123035; Digoxin; ASN3778106; Doxorubicin hydrochloride; PRISTIMERIN; ChemBridge ID #5459675; ChemBridge ID #5113464; STROPHANTHIDINIC ACID LACTONE ACETATE; OUABAIN; ChemBridge ID #6584210; ASN2054045; ASN8911477; ASN655298; AKLAVINE HYDROCHLORIDE; ChemBridge ID #5113312; PUROMYCIN HYDROCHLORIDE; ChemBridge ID #5509904; CONVALLATOXIN; ASN4195110; CYTARABINE; ChemBridge ID #5489051; ChemBridge ID #5686284; ChemBridge ID #5227671; CYCLOHEXIMIDE; ASN5545956; Mitoxantrone dihydrochloride; ChemBridge ID #5625138; Strophanthidin; ChemBridge ID #5557901; ChemBridge ID #5169083; CRINAMINE; PATULIN; ChemBridge ID #5689224; ChemBridge ID #5325790; ChemBridge ID #5686543; ChemBridge ID #5113092; AST 6018098; Digitoxigenin; Lanatoside C; Strophantine octahydrate; ChemBridge ID #5175324; DERRUBONE; 4-NAPHTHOQUINONE; ChemBridge ID #5629242; 5-Nonyloxytryptamine; TEGASEROD MALEATE; ACRISORCIN; ChemBridge ID #5718127; ChemBridge ID #5238658; ChemBridge ID #5524452; ChemBridge ID #5257007; ChemBridge ID #5467697; ChemBridge ID #5526212; ASN5543441; ASN6956358; SARMENTOGENIN; ASN3776811; ASN5443846; ChemBridge ID #5680972; ChemBridge ID #5314590; Ellipticine; ChemBridge ID #5189208; Cycloheximide; ChemBridge ID #5365832; diphenylcyclopropenone; ChemBridge ID #5187143; TRANILAST; ChemBridge ID #5882637; Menadione; ASN6918170; ChemBridge ID #5509444; Tramadol; ASN5303548; ChemBridge ID #5310885; ASN6217614; ChemBridge ID #5353297; ChemBridge ID #5525729; ASN5261664; ASN4456077; ASN5211949; ChemBridge ID #5423993; Indatraline; AST 5544568; ChemBridge ID #5510282; ChemBridge ID #5686316; ChemBridge ID #5113172; ChemBridge ID #5113426; diphenylcyclopropenone; AST 07103543; ChemBridge ID #5175108; ChemBridge ID #5560044; ChemBridge ID #5686326; Quinacrine dihydrochloride dihydrate; Digoxigenin; ChemBridge ID #5217497; ASN5545061; ChemBridge ID #5549342; ChemBridge ID #5397312; ASN2562671; ASN7101546; ChemBridge ID #5128045; ASN6365363; ChemBridge ID #5191891; ChemBridge ID #5417780; ChemBridge ID #5529546; ChemBridge ID #5307689; ASN5113006; ChemBridge ID #5113025; ChemBridge ID #5191886; ASN5260143; L-694 247; ChemBridge ID #5567448; ChemBridge ID #5686465; ChemBridge ID #5376204; ChemBridge ID #5543301; ChemBridge ID #5194467; ChemBridge ID #5652292; ASN9627003; ChemBridge ID #5925531; ChemBridge ID #5230617; HOMOHARRINGTONINE; Etoposide; ChemBridge ID #5560642; TRANILAST; ASN6265182; ChemBridge ID #5691485; ChemBridge ID #5691509; EMICYMARIN; ChemBridge ID #5250077; ASN6917510; AST 5667310; ETOPOSIDE; Indatraline; ChemBridge ID #5135701; ChemBridge ID #5665404; ChemBridge ID #5325782; ChemBridge ID #5222450; ChemBridge ID #5467649; ChemBridge ID #5262233; ChemBridge ID #5286222; NERIIFOLIN; ChemBridge ID #5212518; ChemBridge ID #5217496; ANCITABINE HYDROCHLORIDE; ChemBridge ID #5227831; ChemBridge ID #5283733; ChemBridge ID #5379449; ANTHOTHECOL; ChemBridge ID #5375312; ChemBridge ID #5217961; ChemBridge ID #5255715; ChemBridge ID #6639260; ChemBridge ID #5753084; and combinations thereof.

In other examples, the ATXN2 modulating agent can be Proscillaridin A; ASN5914674; ChemBridge ID #5156626; ChemBridge ID #5352460; ASN5544487; ASN6088278; ASN6291966; ASN4887530; ASN6353460; RESORCINOL MONOACETATE; ASN6414283; ChemBridge ID #5344759; ChemBridge ID #5228409; ChemBridge ID #5268955; ChemBridge ID #5344191; TRIFLUOPERAZINE HYDROCHLORIDE; ASN5544420; TMB-8; THIMEROSAL; ASN5819045; ASN6353027; ASN7185473; AST 6978215; ChemBridge ID #5195242; ASN6087944; Camptothecine (S+); ASN6956162; ASN6353461; ASN4886968; ASN5544494; Trifluridine; ChemBridge ID #5367872; COBALAMINE; ChemBridge ID #5427770; ChemBridge ID #5349968; ASN5114176; ASN6353563; TOMATINE; ASN5262651; ASN4420965; U-37883A; SU1498; PINACIDIL; ChemBridge ID #5223563; ASN6353483; GAMBOGIC ACID; ASN2053976; Emetine dihydrochloride; ChemBridge ID #5354001; ChemBridge ID #5624085; ChemBridge ID #5247688; ChemBridge ID #5378363; Puromycin dihydrochloride; ChemBridge ID #6625505; ASN6088274; ChemBridge ID #5306365; ChemBridge ID #5100075; ChemBridge ID #5425925; ChemBridge ID #5510292; ASN5544434; ChemBridge ID #5545700; ChemBridge ID #5378047; ASN6118229; ChemBridge ID #5510261; STROPHANTHIDIN; ChemBridge ID #5936763; ChemBridge ID #5686303; Niclosamide; ChemBridge ID #5341861; ChemBridge ID #5356272; ASN5114173; ASN3777991; ChemBridge ID #5823090; ChemBridge ID #5453149; MITOXANTHRONE HYDROCHLORIDE; LANATOSIDE C; ASN6957234; ChemBridge ID #5411695; GITOXIGENIN DIACETATE; ChemBridge ID #6538857; ChemBridge ID #5353349; ChemBridge ID #5355005; ASN5114006; Daunorubicin hydrochloride; ACRIFLAVINIUM HYDROCHLORIDE; ChemBridge ID #5553825; ChemBridge ID #5617332; ChemBridge ID #5325791; AMINACRINE; DIGITOXIN; CYMARIN; GITOXIN; ASN3778057; ISOPROPAMIDE IODIDE; ChemBridge ID #5238220; ChemBridge ID #5526179; ASN5021309; Cephaeline dihydrochloride heptahydrate; ChemBridge ID #5276688; PERUVOSIDE; AST6018126; Anisomycin; ChemBridge ID #5268879; ChemBridge ID #5930487; ChemBridge ID #5175328; ChemBridge ID #5244378; and combinations thereof.

In further examples, the ATSN2 modulating agent can be Proscillaridin A; ASN5914674; ChemBridge ID #5156626; ChemBridge ID #5352460; ASN5544487; ASN6088278; ASN6291966; ASN4887530; ASN6353460; RESORCINOL MONOACETATE; ASN6414283; ChemBridge ID #5344759; ChemBridge ID #5228409; ChemBridge ID #5268955; ChemBridge ID #5344191; TRIFLUOPERAZINE HYDROCHLORIDE; ASN5544420; TMB-8; THIMEROSAL; ASN5819045; ASN6353027; ASN7185473; AST 6978215; ChemBridge ID #5195242; ASN6087944; Camptothecine (S+); ASN6956162; ASN6353461; ASN4886968; ASN5544494; Trifluridine; ChemBridge ID #5367872; COBALAMINE; ChemBridge ID #5427770; ChemBridge ID #5349968; ASN5114176; ASN6353563; and combinations thereof.

### EXAMPLES

### Plasmid Cloning

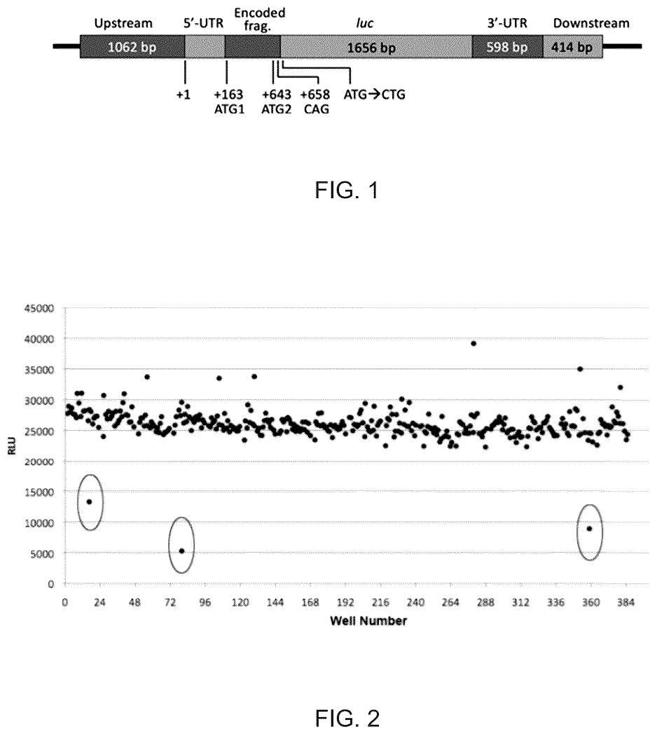

Plasmid pGL2-5A3 contains 1062 bp of ATXN2 upstream sequence, the 162 bp ATXN2 5′-UTR, and an additional 498 bp of the ATXN2 exon 1 sequence encoding through the first CAG of the trinucleotide repeat. This 498 bp tract includes the start codon at +163 as well as the preferred start codon at +643, located 15 bp upstream of the CAG repeat. The firefly luciferase (luc) gene follows, in which the ATG start codon was substituted with CTG, followed by the ATXN2 3′-UTR (598 bp) and 414 bp of ATXN2 downstream sequence. A schematic representation of the ATXN2-luc cassette is shown in FIG. 1. The pGL2-5A3 plasmid was modified to include the hygromycin resistance gene. This was accomplished by amplifying the hygromycin resistance gene insert from pTK-HYG (Clontech) with Pfu polymerase (producing blunt ends) using primers HygSalA (5′-CCTCGGTCGACAGCCCAAGCTTGGCACTG-3′) and HygBluntB (5′-CTTGGAGTGGTGAATCCGTTAGCGAGGTG-3′), cutting pGL2-5A3 with Sal I and Afe I, and ligating in the hygromycin resistance gene insert prepared by digestion with Sal I. The resultant plasmid was designated pGL2h-5A3.

Plasmid pRLh-5A3 is identical to pGL2h-5A3 except Renilla luciferase (Rluc) (with its ATG start codon substituted to CTG) replaces firefly luciferase. First, pRL-5A3 was prepared by amplifying the Rluc insert by PCR from vector pRL-SV40 (Promega) using primers Renl2-A (5′-GCTACTCGAGCTGACTTCGAAAGTTTATGA-3′) and RenlB (5′-CGCTACCGGTTTATTGTTCATTTTTGAGAA-3′). The amplicon was then digested with Xho I and Afe I and ligated into plasmid pGL2-5A3 prepared by Xho I and Afe I digestion to remove the firefly luc insert. pRLh-5A3 with the hygromycin resistance gene was then prepared in the same manner as how the hygromycin gene was inserted into pGL2-5A3, described in the previous paragraph.

Plasmid pGL2h-CMV-luc was prepared using the vector pGL2-Enhancer (Clontech). The CMV insert was amplified from pCMV-MYC (Clontech) with primers CMV-A (5′-GTTGACATTGATTATTGACTA-3′) and CMV-B (5′-GAGCTCTGCTTATATAGA-3′) using Pfu polymerase, and ligated into the Sma I site of pGL2-Enhancer, creating the plasmid pGL2-CMV-luc. Plasmid pGL2-CMV-luc was then modified by the addition of the hygromycin resistance gene in the same manner as it was added into pGL2h-5A3, described above. The resultant plasmid was designated pGL2h-CMV-luc. All inserts were sequenced in both directions to verify proper plasmid construction.

### Assay Cell Lines

The primary screening assay cell line was generated by transfecting HEK-293 cells with pG12h.5A3 and selecting with hygromycin. Transfections were done six separate times resulting in cultures designated H1, H2, H3, S1, S2 and S3. H2 cells were used in the initial pilot screen.

The S2 cell line that expressed a higher level of ATXN2-luc was used in the primary qHTS. Similarly, the counter-screening assay cell line was generated by transfecting HEK-293 cells with pGL2h-CMV-luc and selecting with hygromycin. Two such lines were made, with the resultant cultures designated HC and SC cells. Additionally, HEK-293 cells were transfected with pRLh-5A3 and selecting with hygromycin, with the resultant cell line designated SR cells.

Cultures were maintained as mixed populations of transfected cells (clonal colony selection was not done). Cells lines were tested for the absence of mycoplasma using the Venor GeM Mycoplasma Detection Kit (Sigma).

### Libraries and Compounds

Libraries screened at MSSR: 65,728 compounds were screened from 10 custom and commercially available libraries including compounds from the following sources: Biomol International Enzyme Inhibitors (300), Biomol International Lipid Library (204 compounds), Prestwick Chemicals (1,120 compounds), Microsource Spectrum (2,000 compounds), Asinex Targeted Library (8,505 compounds), Asinex Platinum Collection (19,570 compounds), Biofocus (607 compounds), Chembridge Corp (30,000 compounds), NIH Clinical Collection (606 compounds), UCLA proprietary ES and S (2,816 compounds).

Libraries screened at NCATS: A total of 363,021 compounds were screened including compounds in the following libraries: Sigma Aldrich LOPAC1280 (1,280 compounds), NIH Small Molecular Repository (357,277), NCGC's repurposing collection (2,552 compounds), and NCGC's mechanistically annotated collection (1,912 compounds).

Other compounds used in the study that were not provided by NCATS or sourced from libraries were the following: 17-DMAG (InvivoGen, ant-dlg-25), HSP990 (Santa Cruz Biotechnology, sc-364508), and proscillaridin A (Sigma, R214310). All compound solutions were prepared in DMSO solvent.

### MSSR Pilot Screen

The pilot screen was conducted at the Molecular Screening Shared Resource (MSSR) located at University of California, Los Angeles (UCLA). A reverse plating method was used with H2 cells in 384 well plates. To do so 25 ul media was pre-plated using a Multidrop instrument, 0.5 ul compound was added using a Biomek instrument with a pinning tool, then 25 ul media containing 5,000 cells was added to wells. The final concentration was 10 μM compound in 1% DMSO. Plates were incubated overnight at 37 degrees C. followed by luciferase assays using an automated robotic system for adding 50 ul Bright Glo reagent and recording of RLU values. A total of 197 plates were screened. Using the HTS Corrector software package, data were background corrected and normalized by Z-score transformation to create standardized data with mean of 0 and SD of 1 (Z-score=(X-μ)/σ where u and σ are mean and SD, respectively).

Compounds lowering luciferase expression by >3 SD were considered positive hits and were “cherry picked” for further analysis. Positive hit compounds were retested at two doses (1 and 10 M) using H2 cells, S1 cells and HC cells, with paired MTT viability assays.

Primary qHTS

The primary assay was conducted at the National Center for Advancing Translational Sciences (NCATS) NIH Chemical Genomics Center (NCGC) laboratory. 4 μl of S2 cell suspension in phenol-red free DMEM was dispensed into wells of 1536-well assay plates. After 2 hrs at 37° C. compounds were transferred via a Kalypsys pin tool equipped with a 1536-pin 23 nl slotted pin array. The majority of assays included final concentrations of 57, 11.4, 2.28, 0.46, 0.091, 0.018, 0.0037 M. After incubation at 37° C. for 24 hrs, Gly-Phe-7-Amino-4-trifluoromethylcoumarin (1 μl prepared at 125 uM in PBS) was added and plates incubated for 30 min and imaged with a ViewLux high-throughput CCD imager (PerkinElmer), wherein single end-point fluorescence measurements were acquired to assess cell viability (ex: 405/10 and em: 540/25). Next, SteadyGlo luciferase substrate detection reagent (3 μl) was added to each well and incubated for an additional 5 minutes at room temperature. Luminescence was then measured on the ViewLux imager equipped with clear filters using a 2 sec exposure. All screening operations were performed on a fully integrated robotic system (Kalypsys) containing one RX-130 and two RX-90 anthropomorphic robotic arms (Staubli). Vehicle-only plates, with DMSO being pin-transferred to columns 5-48, were inserted uniformly at the beginning and the end of each library in order to monitor and record any shifts in assay performance. Dose-response curves were fit using the Hill equation, and then curve classes were determined as described, where 1=complete response, 2=incomplete response, 3=single point activity, and 4=inactive.

### Primary Counter-Screen

Compounds evaluated in the primary screen were evaluated in the same manner (viability and luciferase assays) using SC cells expressing CMV-luc. The counter-screen identifies compounds that lower luciferase expression by common transcriptional mechanisms or are luciferase inhibitors. Compounds were considered as active against ATXN2-luc if they inhibited ATXN2-luc in the primary assay (S2 cells) with curve classes −1.1, −1.2, −2.1, or −2.2 and an calculated activity score of >40, did not inhibit CMV-luc in the primary counter-screen (SC cells), and did not alter cell viability in either assay (curve class other than −1.1, −1.2, −2.1, −2.2 in both assays).

### Recombinant Firefly Luc Inhibitor Counter-Screen

Confirmed qHTS active compounds were tested in a biochemical assay to directly measure potential for luciferase inhibition. Briefly, 3 μl of substrate buffer were dispensed into 1536-well assay plates. Compounds were then transferred via Kalypsys pin tool equipped with 1536-pin array. Following addition of compound, 1 μl of recombinant firefly luciferase (10 nM final) was added to initiate the reaction. After 30-minute incubation at room temperature, 2 μl of Kinase-Glo detection reagent was dispensed into each well, followed by an additional 10 min room temperature incubation. End-point measurements of luminescence were acquired using a Viewlux plate reader equipped with clear filters.

### Secondary Assays

Compounds were plated at 2× concentrations in 25 μl in 384 well plates with each compound dose plated in triplicate. An equal volume of S2, H2, or SC cells (24,000 cells/well) suspended in phenol-red free Dulbecco's Modified Eagle Medium (DMEM) with 10% fetal bovine serum and 1× penicillin/streptomycin were added. Cells were grown 24 hrs then assayed first by using the CellTiter-Fluor reagent (20 μl) followed by the addition of Bright-Glo (65 l) luciferase assay, using a multimode plate reader (Beckman DT880). Values were reported as the mean±standard deviation (SD) with n=3. IC50s were determined by the Hill coefficient method using an online tool (https://www.aatbio.com/tools/ic50-calculator).

### RNAi Assays

H-EK-293 cells were cultured in 6-well dishes in DMEM with 10% FBS and 1× pen/strep for 24 hours. Cells were then transfected with vehicle, 50, 100, or 200 nM of a cocktail of four shRNA plasmids targeting expression of the NaK-ATPase a subunit (Santa Cruz Biochemicals, cat. #sc-43956-SH) using Lipofectamine 2000 transfection reagent (ThermoFisher).

### Mice

BAC-ATXN2-Q22 (BAC-Q22) mice were used as previously described. BAC-Q22 are transgenic for the complete human ATXN2 gene with all introns and exons, including 16 kb upstream sequence driving ATXN2 expression and the complete 3′-UTR. The mice used in this study were maintained on a mixed B6;D2 background with backcrossing to wildtype vendor-purchased (Jackson Laboratories) mice every five generations. Mice were treated with 17-DMAG or HSP990 by intraperitoneal (IP) injection, 200-300 μl total volume. 17-DMAG was diluted in 40% DMSO and 0.05% Tween 20 and mice received 100 mg/kg 17-DMAG. HSP990 was diluted in 0.6% DMSO and 0.05% Tween 20 and mice received 4 mg/kg HSP990. Control treated mice included vehicle alone. Mice were treated repeatedly as described in Results. Mouse husbandry and surgical procedures were in accordance to Institutional Animal Care and Use Committee (IACUC) approved protocols.

### Western Blot Assays

Proteins were prepared, separated on precast polyacrylamide gels (Bio-Rad), transferred to Hybond (Amersham) and detected by ECL (Amersham) as previously described.

Antibodies included the following: mouse monoclonal anti-Ataxin-2 antibody (Clone 22/Ataxin-2) (BD Biosciences, 611378), rabbit monoclonal anti-Hsp70 antibody (EPR16892) (Abcam, ab181606), mouse monoclonal anti-β-Actin-peroxidase antibody (clone AC-15) (Sigma-Aldrich, A3854), rabbit polyclonal anti-Staufen antibody (Novus Biologicals, NBP1-33202), mouse monoclonal anti-CHOP (L63F7) antibody (Cell Signaling Technology, 2895), rabbit polyclonal anti-phospho-eIF2a (Ser51) antibody (Cell Signaling Technology, 9721), rabbit polyclonal anti-mTOR antibody (Cell Signaling Technology, 2972), rabbit polyclonal anti-SQSTM1/p62 antibody (Cell Signaling Technology, 5114), rabbit polyclonal anti-LC3B antibody (Novus Biologicals, NB100-2220), rabbit monoclonal anti-BiP antibody (C50B12) (Cell Signaling Technology, 3177). Secondary antibodies included peroxidase-conjugated horse anti-mouse IgG (Vector Laboratories, PI-2000) and peroxidase AffiniPure goat anti-rabbit IgG (Jackson ImmunoResearch Laboratories, 111-035-144).

Quantitative Real Time PCR (qPCR)

Total RNA was extracted from cells cultured in 24-well plates using the RNeasy Mini-Kit (Qiagen Inc.) according to the manufacturer's protocol. DNAse I treated RNAs were used to synthesize cDNA using ProtoScript cDNA synthesis kit (New England Biolabs Inc.). Sets of primers used for qPCR included ATXN2 primers ATXN2-F (5′-AAGATATGGACTCCAGTTATGCAAA-3′) & ATXN2-R (5′-CAAAGCCTCAAGTTCCTCAT-3′), ATP1A2 primers ATP1A2-F (5′-AAGACCCGCCGCAACTCAGTCTTC-3′) & ATP1A2-R (5′-ACCAGGTGACTTTGAGCGGGTACA-3′), GAPDH primers GAPDH-F133 (5′-GAAGATGGTGATGGGATTTCC-3′) & GAPDH-R333 (5′-GAAGGTGAAGGTCGGAGTCAA-3′), and mouse Actb primers Actb-F (5′-CGTCGACAACGGCTCCGGCATG-3′) & Actb-R (5′-GGGCCTCGTCACCCACATAGGAG-3′). qPCR was performed in the Bio-Rad CFX96 instrument (Bio-Rad Inc.) with the Power SYBR Green PCR master mix (Applied Biosystems Inc.). PCR amplification was carried out for 45 cycles. Cycling parameters were denaturation (95° C. for 10 s), annealing (60° C. for 10 s), extension (72° C. for 40 s). The threshold cycle for each sample was chosen from the linear range and converted to a starting quantity by interpolation from a standard curve run on the same plate for each set of primers.

### Results

### MSSR Pilot Screen

Initially, a pilot screen of 65,728 compounds was conducted. In this pilot screen, H2 cells were treated with compounds for 24 hrs at a single 10 μM dose, then assayed for luciferase.

FIG. 2 provides an example of the quality of the raw, untransformed data in this pilot HTS. This chart shows relative luciferase units vs. well number for cells treated with 352 Asinex compounds from one example plate of the pilot screen. Three hits are circled, form right to left these hits are greater than 5, 7, and 6 standard deviations below the mean, respectively. The screen identified 330 hits reducing ATXN2-luc expression by ≥2.7 SD, for an initial hit-rate of 0.51%. The initial hit compounds, their function, and the number of standard deviations from the mean are listed in Table 1.

TABLE 1

| Compound Name | Function if Known | SD from Mean |
|---|---|---|
| Proscillaridin A | cardiac glycoside | −7.684 |
| ASN5914674 | | −7.400 |
| ChemBridge ID# 5156626 | | −6.811 |
| ChemBridge ID# 5352460 | | −6.754 |
| ASN5544487 | | −6.544 |
| ASN6088278 | | −6.506 |
| ASN6291966 | | −6.313 |
| ASN4887530 | | −6.302 |
| ASN6353460 | | −6.253 |
| RESORCINOL | antiseborrheic antipruritic | −6.242 |
| MONOACETATE | | |
| ASN6414283 | | −6.160 |
| ChemBridge ID# 5344759 | | −6.081 |
| ChemBridge ID# 5228409 | | −6.078 |
| ChemBridge ID# 5268955 | | −5.963 |
| ChemBridge ID# 5344191 | | −5.915 |
| TRIFLUOPERAZINE | antipsychotic | −5.856 |
| HYDROCHLORIDE | | |
| ASN5544420 | | −5.770 |
| TMB-8 | Calcium channel blocker | −5.740 |
| THIMEROSAL | antibacterial | −5.645 |
| ASN5819045 | | −5.645 |
| ASN6353027 | | −5.557 |
| ASN7185473 | | −5.458 |
| AST 6978215 | | −5.445 |
| ChemBridge ID#5195242 | | −5.405 |
| ASN6087944 | | −5.358 |
| Camptothecine (S+) | topoisomerase I inhibitor | −5.283 |
| ASN6956162 | | −5.277 |
| ASN6353461 | | −5.258 |
| ASN4886968 | | −5.247 |
| ASN5544494 | | −5.181 |
| Trifluridine | pyrimidine nucleoside | −5.158 |
| | antiviral | |
| ChemBridge ID# 5367872 | | −5.115 |
| COBALAMINE | Vitamin B12 | −5.111 |
| ChemBridge ID# 5427770 | | −5.104 |
| ChemBridge ID# 5349968 | | −5.081 |
| ASN5114176 | | −5.077 |
| ASN6353563 | | −5.041 |
| TOMATINE | antifungal antibacterial | −4.999 |
| ASN5262651 | | −4.983 |
| ASN4420965 | | −4.972 |
| U-37883A | potassium channels | −4.965 |
| SU1498 | Flk-1 kinase inhibitor | −4.885 |
| PINACIDIL | potassium channels | −4.875 |
| ChemBridge ID# 5223563 | | −4.853 |
| ASN6353483 | | −4.849 |
| GAMBOGIC ACID | anti-inflammatory cytotoxic | −4.841 |
| | apoptosis inducer, ETS1 | |
| | inhibitor | |
| ASN2053976 | | −4.841 |
| Emetine dihydrochloride | Calcium channel blocker | −4.840 |
| ChemBridge ID# 5354001 | | −4.823 |
| ChemBridge ID# 5624085 | | −4.780 |
| ChemBridge ID# 5247688 | | −4.779 |
| ChemBridge ID# 5378363 | | −4.754 |
| Puromycin dihydrochloride | | −4.723 |
| ChemBridge ID# 6625505 | | −4.711 |
| ASN6088274 | | −4.705 |
| ChemBridge ID# 5306365 | | −4.667 |
| ChemBridge ID# 5100075 | | −4.663 |
| ChemBridge ID# 5425925 | | −4.635 |
| ChemBridge ID# 5510292 | | −4.627 |
| ASN5544434 | | −4.620 |
| ChemBridge ID# 5545700 | | −4.572 |
| ChemBridge ID# 5378047 | | −4.565 |
| ASN6118229 | | −4.513 |
| ChemBridge ID# 5510261 | | −4.495 |
| STROPHANTHIDIN | cardiac glycoside | −4.451 |
| ChemBridge ID# 5936763 | | −4.444 |
| ChemBridge ID# 5686303 | | −4.416 |
| Niclosamide | anti tenia | −4.393 |
| | worms (tapeworms) & | |
| | cestodes | |
| ChemBridge ID# 5341861 | | −4.381 |
| ChemBridge ID# 5356272 | | −4.370 |
| ASN5114173 | | −4.368 |
| ASN3777991 | | −4.368 |
| ChemBridge ID# 5823090 | | −4.347 |
| ChemBridge ID# 5453149 | | −4.337 |
| MITOXANTHRONE | anticancer | −4.333 |
| HYDROCHLORIDE | | |
| LANATOSIDE C | cardiac glycoside | −4.323 |
| ASN6957234 | | −4.314 |
| ChemBridge ID# 5411695 | | −4.300 |
| GITOXIGENIN | cardiac glycoside | −4.261 |
| DIACETATE | | |
| ChemBridge ID# 6538857 | | −4.243 |
| ChemBridge ID# 5353349 | | −4.237 |
| ChemBridge ID# 5355005 | | −4.219 |
| ASN5114006 | | −4.189 |
| Daunorubicin hydrochloride | cytotoxic antibiotic | −4.155 |
| | anticancer | |
| ACRIFLA VINIUM | intercalating agent | −4.148 |
| HYDROCHLORIDE | preservative | |
| ChemBridge ID# 5553825 | | −4.122 |
| ChemBridge ID# 5617332 | | −4.121 |
| ChemBridge ID# 5325791 | | −4.109 |
| AMINACRINE | antiseptic | −4.103 |
| DIGITOXIN | cardiac glycoside | −4.100 |
| CYMARIN | cardiac glycoside | −4.099 |
| GITOXIN | cardiac glycoside | −4.086 |
| ASN3778057 | | −4.084 |
| ISOPROPAMIDE IODIDE | anticholinergic | −4.073 |
| ChemBridge ID# 5238220 | | −4.072 |
| ChemBridge ID# 5526179 | | −4.069 |
| ASN5021309 | | −4.067 |
| Cephaeline dihydrochloride | maybe serotonin and | −4.055 |
| heptahydrate | dopamine receptors | |
| ChemBridge ID# 5276688 | | −4.047 |
| PERUVOSIDE | cardiac glycoside | −4.038 |
| AST6018126 | | −4.020 |
| Anisomycin | protein synthesis inhibitor | −4.015 |
| ChemBridge ID# 5268879 | | −4.013 |
| ChemBridge ID# 5930487 | | −4.013 |
| ChemBridge ID# 5175328 | | −4.009 |
| ChemBridge ID# 5244378 | | −4.002 |
| ChemBridge ID# 5105504 | | −3.992 |
| ASN3574739 | | −3.988 |
| ChemBridge ID# 5123035 | | −3.988 |
| Digoxin | cardiac glycoside | −3.904 |
| ASN3778106 | | −3.902 |
| Doxorubicin hydrochloride | | −3.897 |
| PRISTIMERIN | anticancer | −3.883 |
| ChemBridge ID# 5459675 | | −3.859 |
| ChemBridge ID# 5113464 | | −3.857 |
| STROPHANTHIDINIC | cardiac glycoside | −3.855 |
| ACID LACTONE | | |
| ACETATE | | |
| OUABAIN | cardiac glycoside | −3.832 |
| ChemBridge ID# 6584210 | | −3.783 |
| ASN2054045 | | −3.780 |
| ASN8911477 | | −3.772 |
| ASN655298 | | −3.750 |
| AKLAVINE | neurotoxin | −3.741 |
| HYDROCHLORIDE | | |
| ChemBridge ID# 5113312 | | −3.737 |
| PUROMYCIN | protein synthesis inhibitor | −3.716 |
| HYDROCHLORIDE | | |
| ChemBridge ID# 5509904 | | −3.706 |
| CONVALLATOXIN | cardiac glycoside | −3.689 |
| ASN4195110 | | −3.685 |
| CYTARABINE | anticancer | −3.681 |
| ChemBridge ID# 5489051 | | −3.678 |
| ChemBridge ID# 5686284 | | −3.675 |
| ChemBridge ID# 5227671 | | −3.673 |
| CYCLOHEXIMIDE | protein synthesis inhibitor | −3.670 |
| ASN5545956 | | −3.669 |
| Mitoxantrone | topoisomerase inhibitor | −3.666 |
| dihydrochloride | | |
| ChemBridge ID# 5625138 | | −3.664 |
| Strophanthidin | cardiac glycoside | −3.662 |
| ChemBridge ID# 5557901 | | −3.659 |
| ChemBridge ID# 5169083 | | −3.653 |
| CRINAMINE | Amaryllidaceae Alkaloid | −3.651 |
| | apoptosis inducing | |
| PATULIN | antibacterial | −3.651 |
| ChemBridge ID# 5689224 | | −3.648 |
| ChemBridge ID# 5325790 | | −3.646 |
| ChemBridge ID# 5686543 | | −3.640 |
| ChemBridge ID# 5113092 | | −3.640 |
| AST 6018098 | | −3.633 |
| Digitoxigenin | cardiac glycoside | −3.629 |
| Lanatoside C | cardiac glycoside | −3.629 |
| Strophantine octahydrate | anticancer agent | −3.624 |
| ChemBridge ID# 5175324 | | −3.623 |
| DERRUBONE | HSP90 inhibitor | −3.621 |
| 4-NAPHTHOQUINONE | from coal tar antibacterial | −3.613 |
| | antitumor | |
| ChemBridge ID# 5629242 | | −3.609 |
| 5-Nonyloxytryptamine | serotonin receptor agonist | −3.607 |
| TEGASEROD MALEATE | serotonin 5-HT4 agonist | −3.606 |
| ACRISORCIN | antifungal | −3.600 |
| ChemBridge ID# 5718127 | | −3.592 |
| ChemBridge ID# 5238658 | | −3.590 |
| ChemBridge ID# 5524452 | | −3.589 |
| ChemBridge ID# 5257007 | | −3.577 |
| ChemBridge ID# 5467697 | | −3.573 |
| ChemBridge ID# 5526212 | | −3.549 |
| ASN5543441 | | −3.547 |
| ASN6956358 | | −3.545 |
| SARMENTOGENIN | cardiac glycoside | −3.539 |
| ASN3776811 | | −3.539 |
| ASN5443846 | | −3.529 |
| ChemBridge ID# 5680972 | | −3.525 |
| ChemBridge ID# 5314590 | | −3.492 |
| Ellipticine | chemotherapeutic | −3.485 |
| ChemBridge ID# 5189208 | | −3.484 |
| Cycloheximide | protein translation | −3.482 |
| ChemBridge ID# 5365832 | | −3.473 |
| diphenylcyclopropenone | used for alopecia | −3.454 |
| ChemBridge ID# 5187143 | | −3.454 |
| TRANILAST | anti-angiogenesis | −3.444 |
| ChemBridge ID# 5882637 | | −3.441 |
| Menadione | vitamin K3 | −3.434 |
| ASN6918170 | | −3.421 |
| ChemBridge ID# 5509444 | | −3.419 |
| Tramadol | u-opioid receptor | −3.415 |
| | noradrenergic serotonergic | |
| ASN5303548 | | −3.413 |
| ChemBridge ID# 5310885 | | −3.411 |
| ASN6217614 | | −3.397 |
| ChemBridge ID# 5353297 | | −3.386 |
| ChemBridge ID# 5525729 | | −3.379 |
| ASN5261664 | | −3.377 |
| ASN4456077 | | −3.367 |
| ASN5211949 | | −3.358 |
| ChemBridge ID# 5423993 | | −3.355 |
| Indatraline | blocks reuptake | −3.347 |
| | of dopamine | |
| | norepinephrine | |
| | serotonin | |
| AST 5544568 | | −3.331 |
| ChemBridge ID# 5510282 | | −3.324 |
| ChemBridge ID# 5686316 | | −3.316 |
| ChemBridge ID# 5113172 | | −3.300 |
| ChemBridge ID# 5113426 | | −3.297 |
| diphenylcyclopropenone | used for alopecia | −3.292 |
| AST 07103543 | | −3.292 |
| ChemBridge ID# 5175108 | | −3.292 |
| ChemBridge ID# 5560044 | | −3.290 |
| ChemBridge ID# 5686326 | | −3.287 |
| Quinacrine dihydrochloride | antimalarial inhibitor of | −3.270 |
| dihydrate | phospholipase A2 | |
| Digoxigenin | cardiac glycoside | −3.264 |
| ChemBridge ID# 5217497 | | −3.264 |
| ASN5545061 | | −3.257 |
| ChemBridge ID# 5549342 | | −3.257 |
| ChemBridge ID# 5397312 | | −3.242 |
| ASN2562671 | | −3.241 |
| ASN7101546 | | −3.240 |
| ChemBridge ID# 5128045 | | −3.227 |
| ASN6365363 | | −3.219 |
| ChemBridge ID#5191891 | | −3.213 |
| ChemBridge ID# 5417780 | | −3.206 |
| ChemBridge ID# 5529546 | | −3.193 |
| ChemBridge ID# 5307689 | | −3.185 |
| ASN5113006 | | −3.178 |
| ChemBridge ID# 5113025 | | −3.177 |
| ChemBridge ID# 5191886 | | −3.165 |
| ASN5260143 | | −3.158 |
| L-694 247 | dopamine receptor agonist | −3.156 |
| ChemBridge ID# 5567448 | | −3.155 |
| ChemBridge ID# 5686465 | | −3.146 |
| ChemBridge ID# 5376204 | | −3.139 |
| ChemBridge ID# 5543301 | | −3.139 |
| ChemBridge ID# 5194467 | | −3.134 |
| ChemBridge ID# 5652292 | | −3.132 |
| ASN9627003 | | −3.127 |
| ChemBridge ID# 5925531 | | −3.122 |
| ChemBridge ID# 5230617 | | −3.120 |
| HOMOHARRINGTONINE | anticancer | −3.117 |
| Etoposide | topoisomerase inhibitor | −3.117 |
| | anticancer agent | |
| ChemBridge ID# 5560642 | | −3.110 |
| TRANILAST | anti-allergies | −3.107 |
| ASN6265182 | | −3.104 |
| ChemBridge ID# 5691485 | | −3.103 |
| ChemBridge ID# 5691509 | | −3.096 |
| EMICYMARIN | cardiac glycoside | −3.080 |
| ChemBridge ID# 5250077 | | −3.080 |
| ASN6917510 | | −3.078 |
| AST 5667310 | | −3.074 |
| ETOPOSIDE | anticancer | −3.073 |
| Indatraline | blocks reuptake | −3.071 |
| | of dopamine | |
| | norepinephrine | |
| | serotonin | |
| ChemBridge ID# 5135701 | | −3.070 |
| ChemBridge ID# 5665404 | | −3.069 |
| ChemBridge ID# 5325782 | | −3.061 |
| ChemBridge ID# 5222450 | | −3.061 |
| ChemBridge ID# 5467649 | | −3.057 |
| ChemBridge ID# 5262233 | | −3.053 |
| ChemBridge ID# 5286222 | | −3.053 |
| NERIIFOLIN | cardiac glycoside | −3.045 |
| ChemBridge ID#5212518 | | −3.043 |
| ChemBridge ID# 5217496 | | −3.034 |
| ANCITABINE | anticancer | −3.031 |
| HYDROCHLORIDE | | |
| ChemBridge ID# 5227831 | | −3.029 |
| ChemBridge ID# 5283733 | | −3.029 |
| ChemBridge ID# 5379449 | | −3.021 |
| ANTHOTHECOL | anti-malarial | −3.020 |
| ChemBridge ID# 5375312 | | −3.015 |
| ChemBridge ID# 5217961 | | −3.006 |
| ChemBridge ID# 5255715 | | −3.004 |
| ChemBridge ID# 6639260 | | −3.004 |
| ChemBridge ID# 5753084 | | −3.002 |
| ASN6536856 | | −2.996 |
| ChemBridge ID# 5104648 | | −2.986 |
| ChemBridge ID# 5564442 | | −2.985 |
| ChemBridge ID# 5203855 | | −2.983 |
| ChemBridge ID# 5766483 | | −2.979 |
| ChemBridge ID# 5481761 | | −2.977 |
| Piperlongumine | anti-platelet | −2.973 |
| | aggregation by | |
| | thromboxane | |
| | A(2) receptor | |
| | antagonism | |
| ASN6466120 | | −2.970 |
| Lycorine hydrochloride | propidium & ethidium like | −2.968 |
| ChemBridge ID# 5340605 | | −2.964 |
| ChemBridge ID# 5680866 | | −2.961 |
| ASN3366855 | | −2.958 |
| IDARUBICIN HCl | antitumor antibiotic | −2.944 |
| | (4-demethoxydaunorubicin) | |
| ChemBridge ID# 5228159 | | −2.943 |
| AST 6353657 | | −2.928 |
| ChemBridge ID# 5248195 | | −2.911 |
| ChemBridge ID# 5572643 | | −2.910 |
| ChemBridge ID# 5660491 | | −2.907 |
| ChemBridge ID# 5473743 | | −2.900 |
| ChemBridge ID# 5269238 | | −2.898 |
| AST 6016979 | | −2.887 |
| ChemBridge ID# 5217960 | | −2.887 |
| ChemBridge ID# 5285643 | | −2.885 |
| ChemBridge ID# 5558368 | | −2.884 |
| ChemBridge ID# 5271226 | | −2.883 |
| ChemBridge ID# 5758142 | | −2.882 |
| ChemBridge ID# 5404874 | | −2.877 |
| ChemBridge ID# 5647469 | | −2.877 |
| ChemBridge ID# 5376918 | | −2.876 |
| ChemBridge ID# 5521810 | | −2.869 |
| ChemBridge ID# 5373059 | | −2.857 |
| ChemBridge ID# 5404983 | | −2.857 |
| ChemBridge ID# 5269666 | | −2.849 |
| ChemBridge ID# 5240459 | | −2.847 |
| ASN4393283 | | −2.846 |
| ChemBridge ID#5194468 | | −2.842 |
| ChemBridge ID# 5543372 | | −2.840 |
| ASN8969408 | | −2.839 |
| ChemBridge ID# 6203241 | | −2.837 |
| U-50488 | Calcium channel blocker | −2.834 |
| ChemBridge ID# 5113192 | | −2.834 |
| ChemBridge ID# 5191873 | | −2.834 |
| ASN7103245 | | −2.833 |
| TENIPOSIDE | anticancer | −2.832 |
| ASN6087888 | | −2.830 |
| ASN9859329 | | −2.827 |
| ASN7115927 | | −2.824 |
| ChemBridge ID# 5249982 | | −2.820 |
| ChemBridge ID# 5401241 | | −2.819 |
| ASN3778030 | | −2.817 |
| CLOFAZIMINE | used in drug combination to | −2.792 |
| | treat leprosy | |
| NICARDIPINE | Calcium channel blocker | −2.782 |
| LOMERIZINE DiHCl | calcium channel blocker | −2.782 |
| ASN5021511 | | −2.762 |
| ASN3270008 | | −2.761 |
| PERIPLOCYMARIN | cardiac glycoside | −2.759 |
| DOXORUBICIN | Chemotherapeutic | −2.751 |
| HYDROCHLORIDE | | |
| PARAROSANILINE | anthelmintic | −2.740 |
| PAMOATE | antischistosomal | |
| Helveticoside | cardiac glycoside | −2.733 |
| RUTILANTINONE | antibiotic | −2.722 |
| SKF-96365 | Calcium channel blocker | −2.721 |
| IDARUBICIN | intercalating agent | −2.719 |
| | preservative | |
| ASN3579998 | | −2.719 |
| AST 6118300 | | −2.717 |
| ASN6918830 | | −2.715 |
| ASN5113007 | | −2.713 |
| ASN5588674 | | −2.695 |
| Sanguinarine | inhibits glutamate | −2.689 |
| | decarboxylase | |
| Cyanocobalamin | one of the B-12 vitamins | −2.681 |

A selection of 155 hit compounds was then rescreened at two compound doses (1 and 10 μM) using H2 cells and S1 cells, and paired MTT assays. Table 2 shows the 155 selected hit compounds, the average of the ATXN2-luc response for the H2 and S1 cells at both dose levels, and the average MTT of the H2 and S1 cells at both dose levels.

TABLE 2

| | Luc | Luc | MTT | MTT |
|---|---|---|---|---|
| | at | at | at | at |
| Compound Name | 1 μm | 10 μm | 1 μm | 10 μm |
| Proscillaridin A | 54 | 22 | 47.75334054 | 44.96512214 |
| ASN5914674 | 211 | 172 | 130.2165999 | 110.7398743 |
| ChemBridge ID# | 117 | 25 | 127.8908177 | 67.21646509 |
| 5156626 | | | | |
| ChemBridge ID# | 108 | 3 | 100.9743094 | 34.04346664 |
| 5352460 | | | | |
| ASN5544487 | 104 | 131 | 116.8195505 | 110.68547 |
| ASN6291966 | 113 | 138 | 126.1362803 | 109.2709592 |
| ASN6353460 | 10 | 11 | 97.45163343 | 84.96585541 |
| RESORCINOL | 120 | 5 | 115.2010237 | 19.91195971 |
| MONOACETATE | | | | |
| ASN6414283 | 32 | 38 | 73.88098713 | 83.04810519 |
| ChemBridge ID# | 35 | 2 | 31.06483332 | 15.23319322 |
| 5344759 | | | | |
| ChemBridge ID# | 33 | 17 | 98.4853144 | 108.0876666 |
| 5228409 | | | | |
| ChemBridge ID# | 76 | 56 | 115.9762845 | 97.84606432 |
| 5268955 | | | | |
| ChemBridge ID# | 5 | 5 | 17.09653918 | 21.95211952 |
| 5344191 | | | | |
| TRIFLUOPERAZINE | 105 | 2 | 88.13490365 | 8.759086103 |
| HYDROCHLORIDE | | | | |
| ASN5544420 | 73 | 52 | 139.3157127 | 112.9160448 |
| TMB-8 | 66 | 53 | 73.92179033 | 52.11928253 |
| ASN5819045 | 99 | 57 | 115.5002472 | 128.774887 |
| ASN6353027 | 21 | 31 | 99.83181987 | 96.51315992 |
| ASN7185473 | 114 | 17 | 105.1226343 | 7.875016853 |
| ChemBridge | 118 | 7 | 112.0863798 | 15.76363477 |
| ID#5195242 | | | | |
| ASN6087944 | 93 | 7 | 108.0332623 | 5.481229347 |
| ASN6956162 | 108 | 49 | 107.2852037 | 31.09203545 |
| ASN6353461 | 19 | 26 | 107.1899962 | 77.86609929 |
| ASN4886968 | 17 | 19 | 129.7405626 | 123.4704715 |
| ASN5544494 | 97 | 72 | 130.2982063 | 127.0339506 |
| Trifluridine | 102 | 71 | 80.32789212 | 53.01695284 |
| ChemBridge ID# | 102 | 85 | 127.5915943 | 149.1900861 |
| 5367872 | | | | |
| COBALAMINE | 126 | 109 | 115.8674759 | 108.0196612 |
| ChemBridge ID# | 110 | 122 | 64.3058371 | 45.14193599 |
| 5427770 | | | | |
| ChemBridge ID# | 8 | 8 | 50.39194722 | 18.6198585 |
| 5349968 | | | | |
| ASN5114176 | 83 | 42 | 116.085093 | 78.50534936 |
| ASN6353563 | 76 | 71 | 102.361618 | 97.49243662 |
| ASN5262651 | 121 | 143 | 108.794922 | 128.774887 |
| ASN4420965 | 132 | 62 | 124.6673652 | 53.04415497 |
| U-37883A | 94 | 17 | 105.4626609 | 56.75724582 |
| SU1498 | 22 | 15 | 86.08114277 | 61.70803362 |
| PINACIDIL | 117 | 17 | 106.72756 | 51.52083565 |
| ChemBridge ID# | 48 | 49 | 89.61741977 | 37.11730741 |
| 5223563 | | | | |
| ASN6353483 | 16 | 17 | 111.4199276 | 103.5857139 |
| ASN2053976 | 19 | 19 | 90.59669648 | 82.34084979 |
| Emetine | 18 | 19 | 59.72227807 | 58.07654916 |
| dihydrochloride | | | | |
| ChemBridge ID# | 69 | 53 | 64.79547546 | 54.74428814 |
| 5354001 | | | | |
| ChemBridge ID# | 98 | 3 | 108.3732889 | 1.944952349 |
| 5624085 | | | | |
| ChemBridge ID# | 83 | 27 | 81.97362103 | 27.61016272 |
| 5247688 | | | | |
| ChemBridge ID# | 36 | 30 | 106.8227675 | 93.33051062 |
| 5378363 | | | | |
| ChemBridge ID# | 43 | 29 | 92.39203711 | 84.05458403 |
| 6625505 | | | | |
| ASN6088274 | 100 | 11 | 81.98722209 | 8.446261599 |
| ChemBridge ID# | 67 | 4 | 55.08431478 | 7.099756127 |
| 5306365 | | | | |
| ChemBridge ID# | 147 | 145 | 111.0662999 | 82.69447749 |
| 5100075 | | | | |
| ChemBridge ID# | 53 | 72 | 102.4976287 | 86.69319072 |
| 5425925 | | | | |
| ChemBridge ID# | 101 | 35 | 92.17442006 | 41.26563235 |
| 5510292 | | | | |
| ASN5544434 | 91 | 67 | 115.6362578 | 120.6142478 |
| ChemBridge ID# | 48 | 26 | 116.1258962 | 76.72360979 |
| 5545700 | | | | |
| ASN6118229 | 109 | 127 | 112.970449 | 71.14717299 |
| ChemBridge ID# | 93 | 24 | 103.6673203 | 30.83361521 |
| 5510261 | | | | |
| STROPHANTHIDIN | 16 | 15 | 40.1095418 | 45.52276582 |
| ChemBridge ID# | 23 | 30 | 73.56816263 | 119.3765508 |
| 5936763 | | | | |
| ChemBridge ID# | 92 | 67 | 105.0818311 | 85.46909483 |
| 5686303 | | | | |
| ChemBridge ID# | 87 | 24 | 117.2275825 | 41.22482916 |
| 5341861 | | | | |
| ChemBridge ID# | 120 | 4 | 100.8110966 | 11.11207041 |
| 5356272 | | | | |
| ASN3777991 | 123 | 139 | 121.9335511 | 76.72360979 |
| ASN5114173 | 83 | 52 | 111.8415606 | 106.5643472 |
| ChemBridge ID# | 98 | 49 | 100.1718465 | 116.3843164 |
| 5823090 | | | | |
| ChemBridge ID# | 107 | 122 | 91.71198384 | 117.7036198 |
| 5453149 | | | | |
| LANATOSIDE C | 39 | 15 | 38.04217986 | 35.97481792 |
| ChemBridge ID# | 105 | 140 | 123.375264 | 123.3888651 |
| 5411695 | | | | |
| GITOXIGENIN | 27 | 21 | 39.68790877 | 40.29995671 |
| DIACETATE | | | | |
| ChemBridge ID# | 113 | 10 | 85.4010895 | 118.3020667 |
| 6538857 | | | | |
| ChemBridge ID# | 32 | 31 | 113.9089225 | 101.0151126 |
| 5353349 | | | | |
| ChemBridge ID# | 102 | 122 | 126.9523442 | 97.94127178 |
| 5355005 | | | | |
| ASN5114006 | 100 | 68 | 120.3694286 | 114.3577577 |
| ChemBridge ID# | 11 | 15 | 96.37714926 | 97.68285154 |
| 5553825 | | | | |
| ChemBridge ID# | 85 | 12 | 110.9982945 | 37.82456281 |
| 5617332 | | | | |
| ChemBridge ID# | 133 | 112 | 115.881077 | 100.8791019 |
| 5325791 | | | | |
| DIGITOXIN | 27 | 19 | 34.26108369 | 49.05904282 |
| CYMARIN | 14 | 16 | 54.18664446 | 59.31424611 |
| GITOXIN | 21 | 12 | 28.73905114 | 40.27275458 |
| ASN3778057 | 117 | 122 | 99.31497939 | 69.2566249 |
| ChemBridge ID# | 31 | 38 | 124.9937908 | 88.65174413 |
| 5238220 | | | | |
| ChemBridge ID# | 105 | 115 | 122.736014 | 86.02673851 |
| 5526179 | | | | |
| ASN5021309 | 97 | 85 | 103.7081235 | 105.0274268 |
| Cephaeline | 16 | 17 | 59.13743226 | 62.10246451 |
| dihydrochloride | | | | |
| heptahydrate | | | | |
| ChemBridge ID# | 78 | 8 | 73.58176369 | 46.10761163 |
| 5276688 | | | | |
| PERUVOSIDE | 15 | 12 | 47.48131923 | 60.00790044 |
| ChemBridge ID# | 85 | 79 | 107.2035973 | 109.6109859 |
| 5268879 | | | | |
| ChemBridge ID# | 81 | 39 | 114.8745982 | 124.4769503 |
| 5930487 | | | | |
| ChemBridge ID# | 93 | 23 | 91.24954762 | 27.29733821 |
| 5175328 | | | | |
| ChemBridge ID# | 97 | 3 | 164.1240559 | 18.86467768 |
| 5244378 | | | | |
| ChemBridge ID# | 30 | 37 | 136.255473 | 92.63685629 |
| 5105504 | | | | |
| ASN3574739 | 218 | 283 | 78.31493444 | 72.398471 |
| Digoxin | 47 | 12 | 78.69576427 | 73.48655623 |
| ASN3778106 | 123 | 139 | 121.076684 | 86.97881309 |
| ChemBridge ID# | 100 | 72 | 82.44965832 | 74.1666095 |
| 5459675 | | | | |
| ChemBridge ID# | 57 | 55 | 109.8558051 | 44.3530742 |
| 5113464 | | | | |
| STROPHAN- | 37 | 20 | 43.59141453 | 36.62766906 |
| THIDINIC | | | | |
| ACID LACTONE | | | | |
| ACETATE | | | | |
| OUABAIN | 14 | 12 | 51.75205376 | 60.40233134 |
| ChemBridge ID# | 87 | 91 | 116.4387207 | 89.31819633 |
| 6584210 | | | | |
| ASN2054045 | 33 | 46 | 134.3785259 | 118.7236997 |
| ASN8911477 | 219 | 250 | 103.4497033 | 87.30523866 |
| ChemBridge ID# | 80 | 56 | 108.2780815 | 44.54348911 |
| 5113312 | | | | |
| ChemBridge ID# | 107 | 103 | 90.3926805 | 99.36938365 |
| 5509904 | | | | |
| CONVALLATOXIN | 12 | 11 | 62.15686877 | 70.0046835 |
| ASN4195110 | 112 | 89 | 87.04681842 | 38.72223313 |
| ChemBridge ID# | 128 | 92 | 132.7055949 | 126.3130942 |
| 5489051 | | | | |
| ChemBridge ID# | 49 | 40 | 103.8713363 | 80.16467933 |
| 5686284 | | | | |
| ChemBridge ID# | 111 | 149 | 89.2909942 | 41.86407923 |
| 5227671 | | | | |
| ASN5545956 | 92 | 101 | 173.3591793 | 106.0883099 |
| ChemBridge ID# | 47 | 29 | 43.44180282 | 38.00137666 |
| 5625138 | | | | |
| ChemBridge ID# | 43 | 45 | 39.44308959 | 37.13090848 |
| 5557901 | | | | |
| ChemBridge ID# | 101 | 125 | 139.9821649 | 123.8921045 |
| 5169083 | | | | |
| ChemBridge ID# | 31 | 33 | 135.5074144 | 94.58180863 |
| 5689224 | | | | |
| ChemBridge ID# | 123 | 119 | 131.4951001 | 121.4711149 |
| 5325790 | | | | |
| ChemBridge ID# | 90 | 72 | 107.1899962 | 68.5901727 |
| 5113092 | | | | |
| ChemBridge ID# | 59 | 61 | 55.51954887 | 35.45797744 |
| 5686543 | | | | |
| Digitoxigenin | 38 | 13 | 81.64719546 | 74.84666277 |
| Lanatoside C | 39 | 15 | 35.00914228 | 35.63479129 |
| ChemBridge ID# | 88 | 21 | 104.3609746 | 30.18076407 |
| 5175324 | | | | |
| DERRUBONE | 11 | 15 | 107.1627941 | 101.0695168 |
| ChemBridge ID# | 110 | 58 | 95.80590452 | 41.74166964 |
| 5629242 | | | | |
| 5-Nonyloxytryptamine | 111 | 5 | 114.0585343 | 23.67945482 |
| TEGASEROD | 105 | 8 | 106.1291131 | 14.21311332 |
| MALEATE | | | | |
| ChemBridge ID# | 71 | 49 | 97.91406965 | 91.37195721 |
| 5718127 | | | | |
| ChemBridge ID# | 95 | 16 | 111.8143585 | 20.89123642 |
| 5238658 | | | | |
| ChemBridge ID# | 177 | 184 | 105.6122727 | 93.62973406 |
| 5524452 | | | | |
| ChemBridge ID# | 83 | 47 | 45.88999458 | 31.09203545 |
| 5257007 | | | | |
| ChemBridge ID# | 103 | 34 | 63.44896999 | 12.54018228 |
| 5467697 | | | | |
| ChemBridge ID# | 135 | 86 | 107.135592 | 74.96907236 |
| 5526212 | | | | |
| ASN5543441 | 112 | 79 | 97.98207498 | 66.75402887 |
| ASN6956358 | 104 | 82 | 102.8376553 | 90.20226558 |
| SARMENTOGENIN | 53 | 24 | 45.80838819 | 41.23843022 |
| ASN3776811 | 102 | 131 | 92.97688292 | 109.7061933 |
| ASN5443846 | 110 | 101 | 103.1096766 | 92.74566481 |
| ChemBridge ID# | 127 | 147 | 111.2431137 | 96.47235672 |
| 5680972 | | | | |
| ChemBridge ID# | 53 | 42 | 122.4367905 | 123.7016896 |
| 5314590 | | | | |
| ChemBridge ID# | 115 | 50 | 142.5255641 | 89.26379207 |
| 5882637 | | | | |
| Menadione | 115 | 35 | 152.7263631 | 103.2728894 |
| Tramadol | 107 | 134 | 128.2444454 | 112.8480394 |
| ASN5303548 | 89 | 93 | 112.0863798 | 93.16729783 |
| AST 07103543 | 16 | 19 | 133.7392759 | 128.7884881 |
| Digoxigenin | 90 | 22 | 60.40233134 | 31.56807274 |
| ChemBridge ID# | 103 | 55 | 113.5688959 | 37.56614257 |
| 5217497 | | | | |
| L-694 247 | 110 | 6 | 107.2716026 | 16.36208165 |
| EMICYMARIN | 115 | 31 | 111.923167 | 53.4249848 |
| Indatraline | 107 | 6 | 116.506726 | 13.49225685 |
| NERIIFOLIN | 17 | 17 | 30.79281201 | 36.11082858 |
| ChemBridge | 92 | 109 | 68.5901727 | 97.75085687 |
| ID#5212518 | | | | |
| ChemBridge | 119 | 77 | 129.3461317 | 59.30064505 |
| ID#5194468 | | | | |
| U-50488 | 88 | 68 | 81.22556243 | 55.60115526 |
| NICARDIPINE | 112 | 61 | 139.5197286 | 87.04681842 |
| LOMERIZINE DiHCl | 102 | 55 | 100.9471072 | 37.60694577 |
| PERIPLOCYMARIN | 75 | 17 | 58.70219817 | 37.10370635 |
| Helveticoside | 118 | 41 | 98.96135169 | 37.06290315 |
| SKF-96365 | 104 | 57 | 115.6090557 | 90.0390528 |
| Sanguinarine | 127 | 160 | 102.8784585 | 113.9497257 |
| Cyanocobalamin | 110 | 140 | 151.5430704 | 134.8545632 |

Among these compounds, 43 were annotated, including 21 cardiac glycosides, 6 calcium channel blockers, 3 topoisomerase inhibitors, 1 HSP90 inhibitor. Some of these annotated compounds also appeared as ATXN2-luc inhibitors in the primary qHTS (see below). Of the 155 compounds, 12 were selected that lowered ATXN2-luc by >2.7 SD but did not reduce cell viability (MTT assay) by >65% at either 1 M or 10 M, to further evaluate for modifying CMV-luc expression. These 12 compounds were: lomerizine, trifluoperazine HCl, ASN 05544420, ASN 03574739, ASN 05543441, ASN 05819045, Chembridge 5553825, Chembridge 5689224, Chembridge 6625505, Chembridge 5228409, Chembridge 5718127, and Chembridge 5105504. Testing for CMV-luc modification determines performance of the assays for detecting compounds that function as either luciferase inhibitors or inhibitors of generalized transcription/translation. Among these 12 compounds, four compounds had activity specific against ATXN2-luc (ASN 5544420, ASN 5819045, ChemBridge 5228409, and ChemBridge 5718127).

ChemBridge 5553825 was a potent CMV-luc inhibitor that was later used for further assessing the quality of the S2 primary screening cell line (see below). Multi-concentration testing was performed for ChemBridge 5228409 and ChemBridge 5718127 using ATXN2-luc and CMV-luc, the primary and counterscreens respectively, with matched MTT assays performed for viability assessment. ChemBridge 5228409 or ChemBridge 5718127 were not evaluated further.

### Quality Metrics of the S2 Primary Screening Cell Line

Quality metrics were determined for high-throughput screening for the S2 cell line. Among six HEK-293 cell line clones stably expressing ATXN2-luc (S1, S2, S3, H1, H2, H3), the line designated S2 was selected for the primary screen as it expressed the highest level of ATXN2-luc.

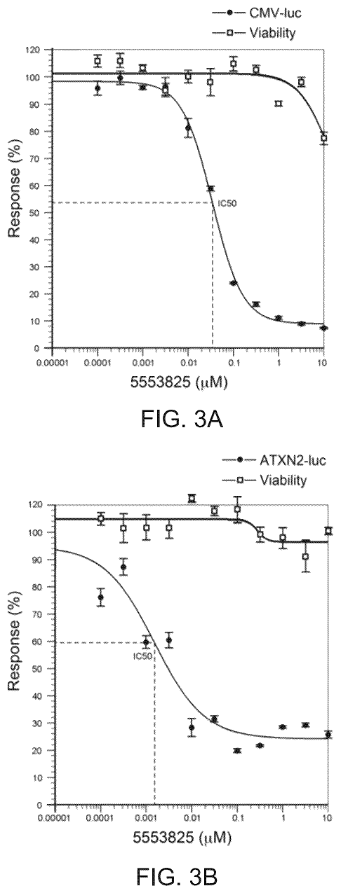

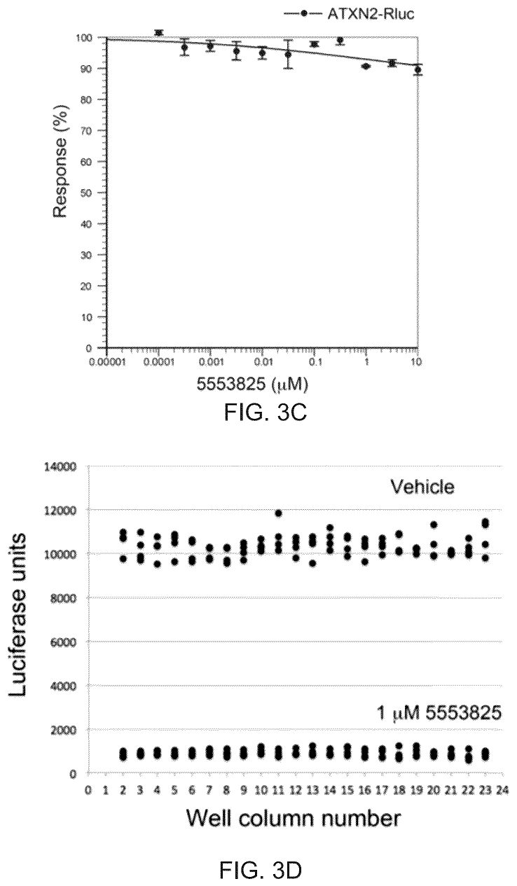

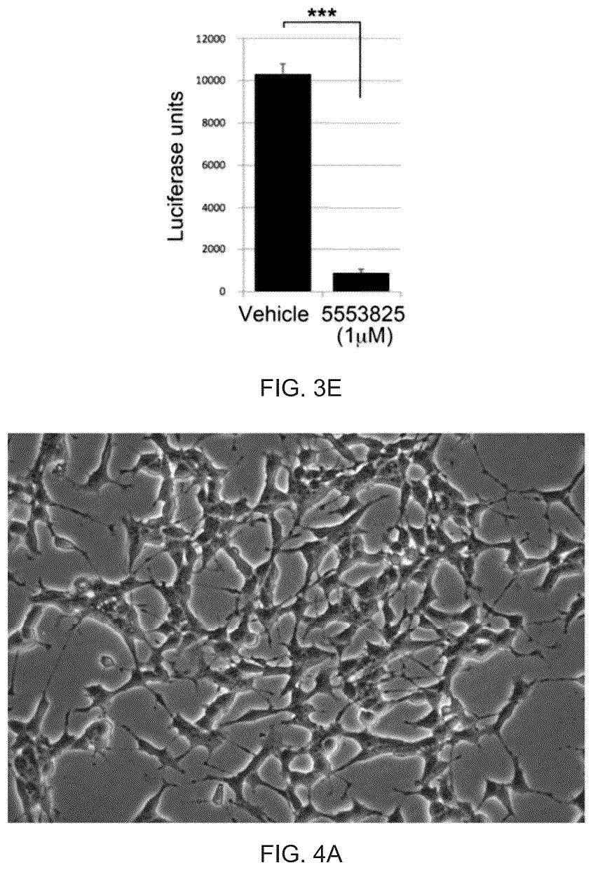

The ChemBridge 5553825 compound, a potent luciferase inhibitor identified in the pilot screen, was used to assess quality metrics for S2. ChemBridge 5553825 inhibited CMV-luc in a dose-dependent manner in HEK-293 cells stably transfected with CMV-luc (SC cells) without inhibiting cell abundance determined by paired MTT assays (FIG. 3A). ChemBridge 5553825 used in M2-log doses inhibited CMV-luc activity in SC cells (IC50=35 nM). S2 cells were also used to demonstrate that ChemBridge 5553825 inhibited ATXN2-luc in a dose-dependent manner without altering cell viability (FIG. 3B, IC50=2 nM), but not ATXN2-Rluc (FIG. 3C). Collectively, these data are consistent with ChemBridge 5553825 as a firefly luciferase inhibitor rather than an inhibitor of ATXN2-luc or CMV-luc transcription or translation.

88 replicate luciferase assays per condition were then performed in a 384 well plate using S2 cells treated with ChemBridge 5553825 vs vehicle (1% DMSO in phenol red-free DMEM, 6000 HEK-293/ATXN2-luc (S1) cells/well were treated with vehicle (1% DMSO) or 1 μM ChemBridge 5553825 in 50 μl total volume in a 384 well plate (n=96 per condition)) (FIG. 3D). After 48 hr treatment, the luciferase unit means and SDs for cells treated with 1 M ChemBridge 5553825 or vehicle were 888149 and 10,321±464, respectively (FIG. 3E). Using known and accepted methods, the Z′-factor was computed as Z′-factor=0.8, and the signal/noise (S/N) ratio was computed as S/N=30.0, signal/background (S/B)=11.6, and coefficient of variation (Cv)=8.6% (p<0.001, Student's t-test. The Z′-factor was calculated as 1-[(3(σexp+σcont))/|μexp-μcont|] where σ and μ are SD and mean, respectively).

### NCGC Pilot Screen Optimization and Miniaturization

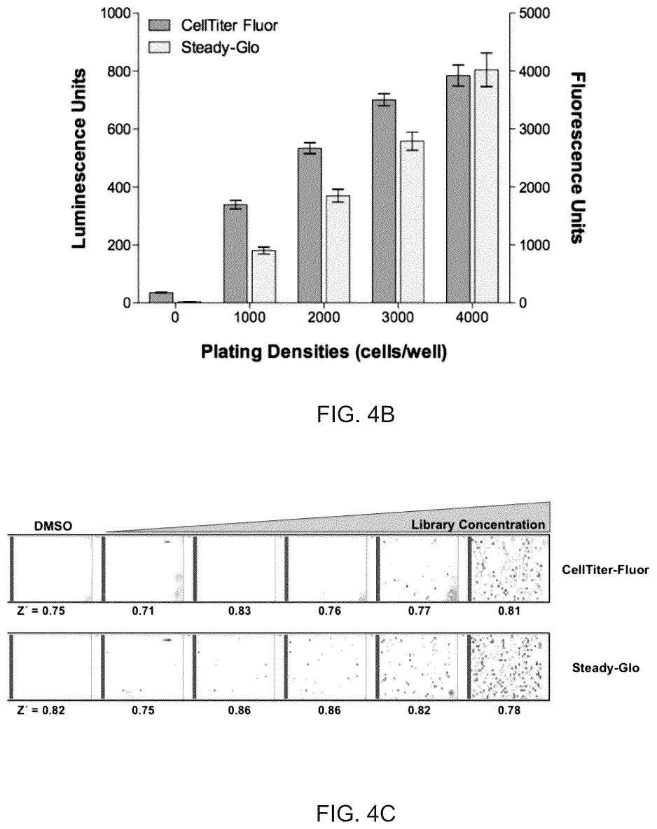

Screening to optimize the assay in 1536-well format was conducted at the NCGC laboratory using S2 cells. A validation screen was performed at four concentrations (ranging from 460 nM to 57 uM) using the LOPAC1280 library, a well characterized set of pharmacologically active molecules. Cytotoxicity was determined using a multiplexed viability assay (CellTiter-Fluor). Assay optimizations demonstrated acceptable cellular densities, linear signal increase with cell number plated, and Z′-factors>0.7 for any library concentration. FIG. 4A shows S2 cells plated at 1,000 cells/well in a well of a 1536 well plate showing subconfluent density at time of assay. FIG. 4B shows the effect of increasing S2 cell abundance on readout signals. Luciferase expression (Steady-Glo) and a compatible same—well-assay measure of cell abundance (CellTiter Fluor) increased linearly with increasing numbers of cells plated. FIG. 4C shows a preliminary miniaturized compound screen using S2 with compound diluent alone (DMSO) or 5 doses of LOPAC library compound (Sigma). Cells were plated 1000 cells/well in 1536 well plates. Plates were assayed for ATXN2-luc expression (Steady-Glo) and compound effect on cell abundance (CellTiter-Fluor), with increases shown for each well indicated by red, and decreases indicated by blue. Numerous ATXN2-luc changes were observed for compounds not altering cell abundance. Z′-score values were all >0.7. Pilot screening of the LOPAC1280 library identified 37 unique active compounds among 58 hits (redundancies occurred) with curve classes between −1.1 and −3. Seven of the annotated compounds were cardiac glycosides (including ouabain and dihydroouabain that were both observed twice), all with curve class −1.1. Furthermore, four other non-annotated compounds (MLS001076487, MLS002153278, MLS001148144, MLS002702983) with cardiac glycoside structures were included among the list of 58 lead compounds. These 58 lead compounds are listed in Table 3S:

TABLE 3S

| | Curve | |
|---|---|---|
| Compound Name | Class | Structure |
| Ouabain | −1.1 | 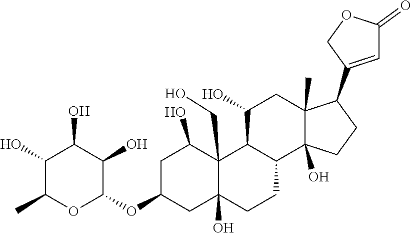 [SMILES: C[C@@H]1O[C@@H](O[C@H]2C[C@@H](O)[C@]3(CO)[C@H]4[C@H](O)C[C@]5(C)[C@@H](C6=CC(=O)OC6)CC[C@]5(O)[C@@H]4CC[C@]3(O)C2)[C@H](O)[C@H](O)[C@H]1O] |
| Ouabain | −1.1 | 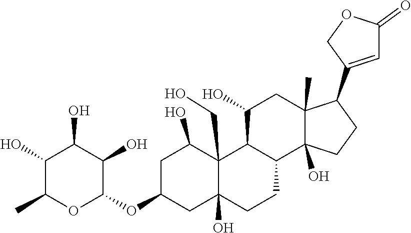 [SMILES: C[C@@H]1O[C@@H](O[C@H]2C[C@@H](O)[C@]3(CO)[C@H]4[C@H](O)C[C@]5(C)[C@@H](C6=CC(=O)OC6)CC[C@]5(O)[C@@H]4CC[C@]3(O)C2)[C@H](O)[C@H](O)[C@H]1O] |
| Strophantine octahydrate | −1.1 | 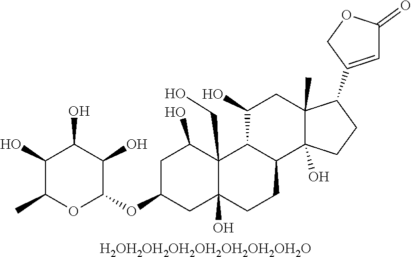 |
| Ouabain | −1.1 | 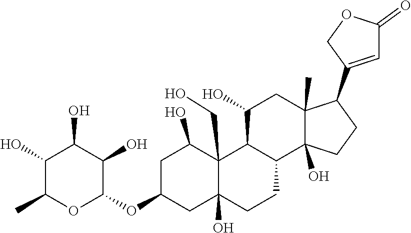 [SMILES: C[C@@H]1O[C@@H](O[C@H]2C[C@@H](O)[C@]3(CO)[C@H]4[C@H](O)C[C@]5(C)[C@@H](C6=CC(=O)OC6)CC[C@]5(O)[C@@H]4CC[C@]3(O)C2)[C@H](O)[C@H](O)[C@H]1O] |
| Dihydroouabain | −1.2 | 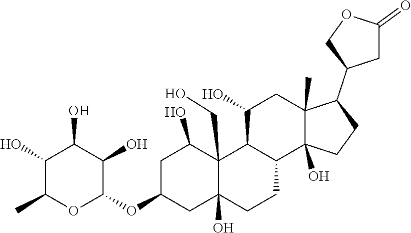 [SMILES: C[C@@H]1O[C@@H](O[C@H]2C[C@@H](O)[C@]3(CO)[C@H]4[C@H](O)C[C@]5(C)[C@@H]([C@H]6COC(=O)C6)CC[C@]5(O)[C@@H]4CC[C@]3(O)C2)[C@H](O)[C@H](O)[C@H]1O] |
| Lanatoside B | −1.1 | 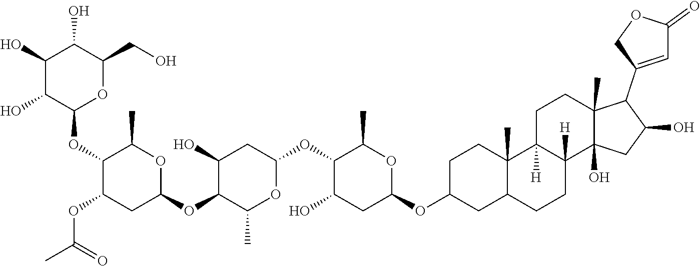 |
| Dihydroouabain | −1.2 | 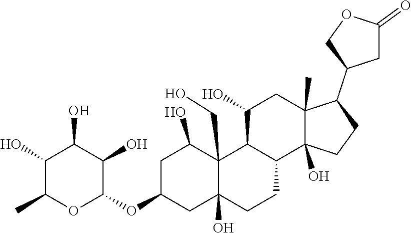 [SMILES: C[C@@H]1O[C@@H](O[C@H]2C[C@@H](O)[C@]3(CO)[C@H]4[C@H](O)C[C@]5(C)[C@@H]([C@H]6COC(=O)C6)CC[C@]5(O)[C@@H]4CC[C@]3(O)C2)[C@H](O)[C@H](O)[C@H]1O] |
| Dihydroouabain | −1.1 | 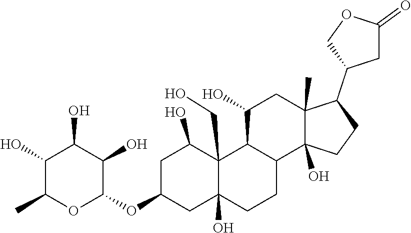 |
| Dihydroouabain | −1.1 | 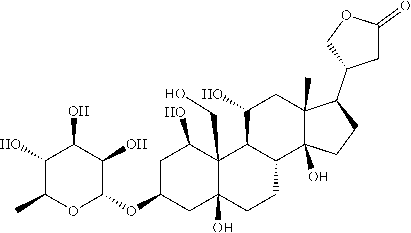 [SMILES: C[C@@H]1O[C@@H](O[C@H]2C[C@@H](O)[C@]3(CO)[C@H]4[C@H](O)C[C@]5(C)[C@@H]([C@@H]6COC(=O)C6)CC[C@]5(O)[C@@H]4CC[C@]3(O)C2)[C@H](O)[C@H](O)[C@H]1O] |
| Thioguanosine | −1.2 | 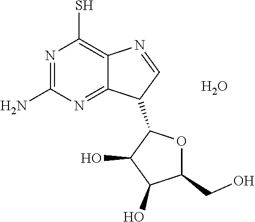 [SMILES: Nc1nc(S)c2c(n1)C([C@@H]1O[C@@H](CO)[C@@H](O)[C@H]1O)C=N2 ; O] |
| Helveticoside | −1.1 | 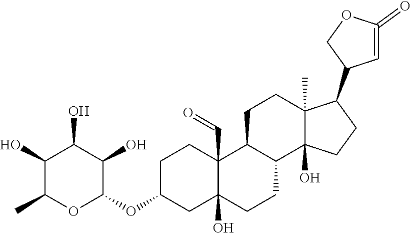 |
| Prazosin hydrochloride | −3 | 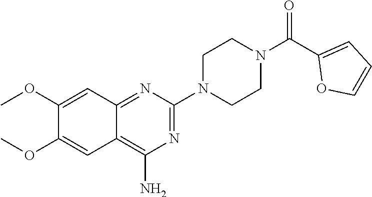 [SMILES: COc1cc2nc(N3CCN(C(=O)c4ccco4)CC3)nc(N)c2cc1OC] |
| A-134974 dihydrochloride hydrate | −2.1 | 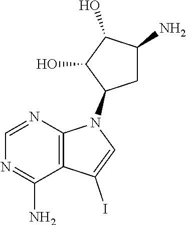 [SMILES: Nc1ncnc2c1c(I)cn2[C@@H]1C[C@H](N)[C@@H](O)[C@H]1O] |
| Ouabain | −2.3 | 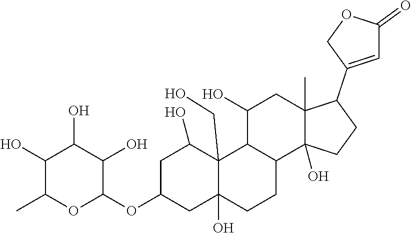 [SMILES: CC1OC(OC2CC(O)C3(CO)C4C(O)CC5(C)C(C6=CC(=O)OC6)CCC5(O)C4CCC3(O)C2)C(O)C(O)C1O] |
| VER-3323 hemifumarate salt | −2.2 | 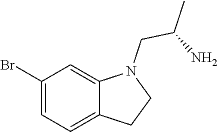 |
| Amoxapine | −2.2 | 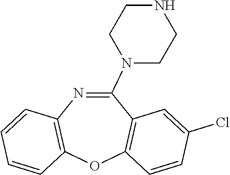 [SMILES: Clc1ccc2c(c1)C(N1CCNCC1)=Nc1ccccc1O2] |
| Fluoxetine hydrochloride | −2.1 | 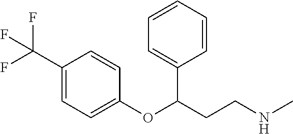 [SMILES: CNCCC(Oc1ccc(C(F)(F)F)cc1)c1ccccc1] |
| A-134974 dihydrochloride hydrate | −2.1 | 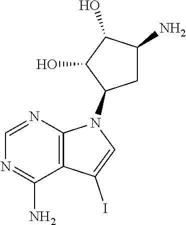 [SMILES: Nc1ncnc2c1c(I)cn2[C@@H]1C[C@H](N)[C@@H](O)[C@H]1O] |
| Desipramine hydrochloride | −2.2 | 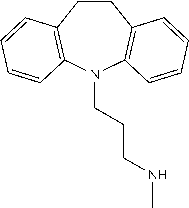 [SMILES: CNCCCN1c2ccccc2CCc2ccccc21] |
| CNS-1102 | −2.2 | 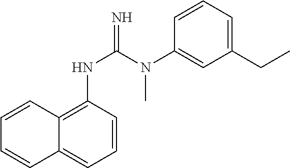 [SMILES: CCc1cccc(N(C)C(=N)Nc2cccc3ccccc23)c1] |
| Fluoxetine hydrochloride | −2.1 | 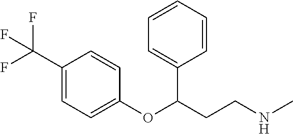 [SMILES: CNCCC(Oc1ccc(C(F)(F)F)cc1)c1ccccc1] |
| Naltrindole hydrochloride | −2.2 | 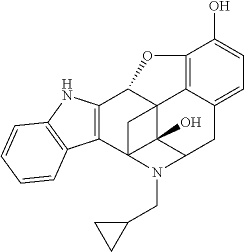 |
| Desipramine hydrochloride | −2.2 | 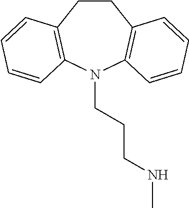 [SMILES: CNCCCN1c2ccccc2CCc2ccccc21] |
| L-687,384 hydrochloride | −2.2 | 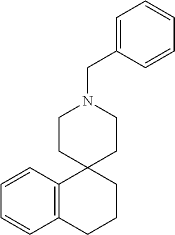 [SMILES: c1ccc(CN2CCC3(CCCc4ccccc43)CC2)cc1] |
| (±)-Propranolol hydrochloride | −2.2 | 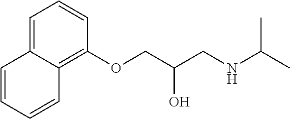 [SMILES: CC(C)NCC(O)COc1cccc2ccccc12] |
| S-(+)-Fluoxetine hydrochloride | −2.2 | 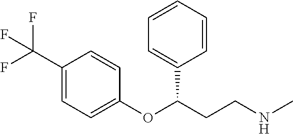 [SMILES: CNCC[C@H](Oc1ccc(C(F)(F)F)cc1)c1ccccc1] |
| VER-3323 hemifumarate salt | −2.2 | 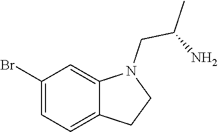 |
| Benzeneethanamine, 4-bromo-2,5- dimethoxy-alpha- methyl-, hydrobromide (1:1) | −2.4 | 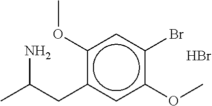 |
| Quinidine sulfate | −2.2 | 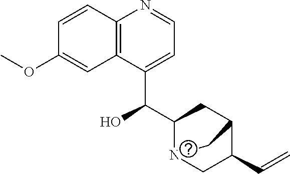 |
| S-(+)-Fluoxetine hydrochloride | −2.2 | 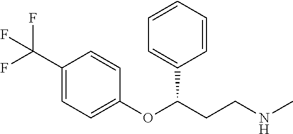 [SMILES: CNCC[C@H](Oc1ccc(C(F)(F)F)cc1)c1ccccc1] |
| (+)-Phenazocine hydrobromide | −2.4 | 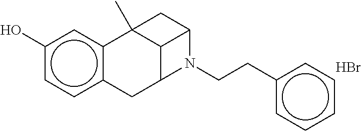 |
| (S)-Propranolol hydrochloride | −2.2 | 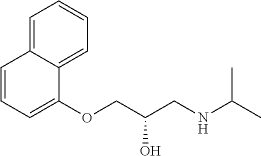 [SMILES: CC(C)NC[C@H](O)COc1cccc2ccccc12] |
| Quinidine sulfate | −2.2 | 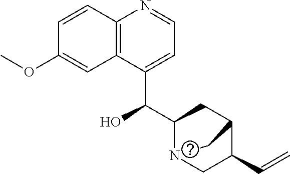 |
| Biperiden hydrochloride | −2.2 | 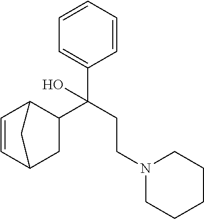 [SMILES: OC(CCN1CCCCC1)(c1ccccc1)C1CC2C=CC1C2] |
| Naltrindole hydrochloride | −2.2 | 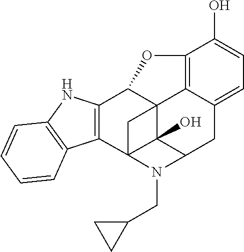 |
| CNS-1102 | −2.2 | 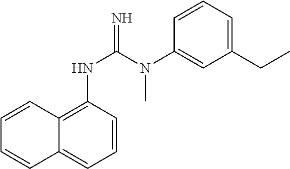 [SMILES: CCc1cccc(N(C)C(=N)Nc2cccc3ccccc23)c1] |
| 1-(5- Isoquinolinylsulfonyl)- 2- methylpiperazine dihydrochloride | −2.2 | 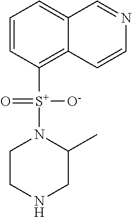 [SMILES: CC1CNCCN1[S+](=O)([O-])c1cccc2cnccc12] |
| 1-(5- Isoquinolinylsulfonyl)- 2- methylpiperazine dihydrochloride | −2.1 | 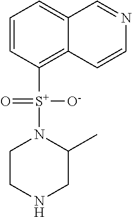 [SMILES: CC1CNCCN1[S+](=O)([O-])c1cccc2cnccc12] |
| | −3 | 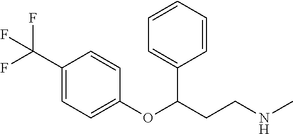 [SMILES: CNCCC(Oc1ccc(C(F)(F)F)cc1)c1ccccc1] |
| (±)-Propranolol hydrochloride | −2.2 | 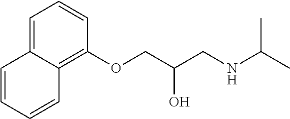 [SMILES: CC(C)NCC(O)COc1cccc2ccccc12] |
| Biperiden hydrochloride | −2.2 | 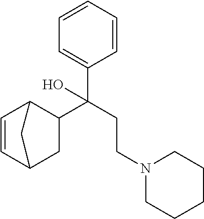 [SMILES: OC(CCN1CCCCC1)(c1ccccc1)C1CC2C=CC1C2] |
| Roscovitine | −2.2 | 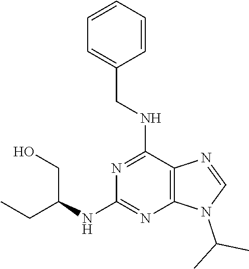 [SMILES: CC[C@@H](CO)Nc1nc(NCc2ccccc2)c2ncn(C(C)C)c2n1] |
| S-(+)-2,5- Dimethoxy-4- bromoamphetamine hydrochloride | −2.2 | 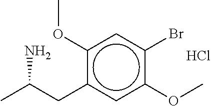 |
| Tomoxetine | −2.2 | 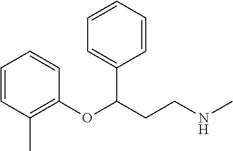 [SMILES: CNCCC(Oc1ccccc1C)c1ccccc1] |
| (S)-Propranolol hydrochloride | −2.2 | 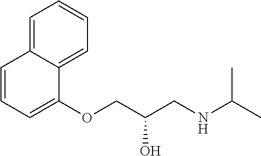 [SMILES: CC(C)NC[C@H](O)COc1cccc2ccccc12] |
| Protriptyline hydrochloride | −2.2 | 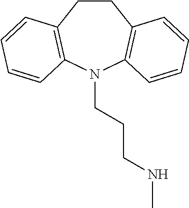 [SMILES: CNCCCN1c2ccccc2CCc2ccccc21] |
| (−)-alpha-Methadol hydrochloride | −2.2 | 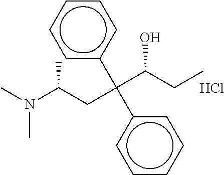 |
| Roscovitine | −2.1 | 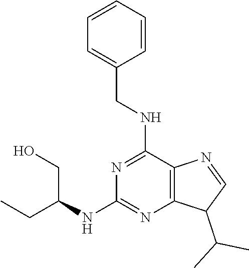 [SMILES: CC[C@@H](CO)Nc1nc(NCc2ccccc2)c2c(n1)C(C(C)C)C=N2] |
| S(+)-Isoproterenol (+)-bitartrate | −3 | 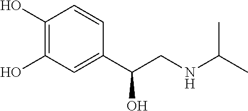 |
| BRL 52537 hydrochloride | −3 | 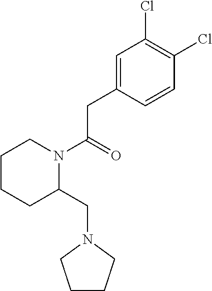 [SMILES: O=C(Cc1ccc(Cl)c(Cl)c1)N1CCCCC1CN1CCCC1] |
| (+)-alpha-Methadol hydrochloride | −2.2 | 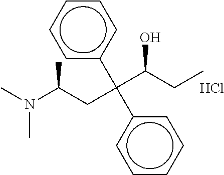 |
| (−)-Phenazocine hydrobromide | −2.2 | 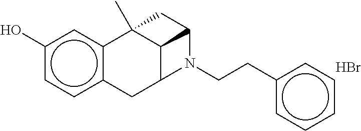 |
| Quinine sulfate | −3 | 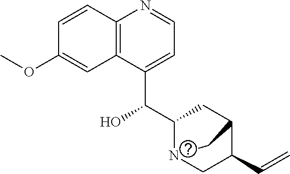 |
| Prazosin hydrochloride | −3 | 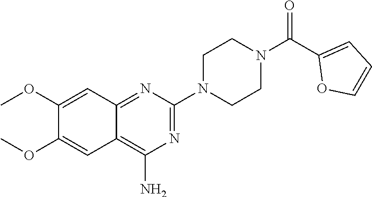 [SMILES: COc1cc2nc(N3CCN(C(=O)c4ccco4)CC3)nc(N)c2cc1OC] |
| | −2.2 | 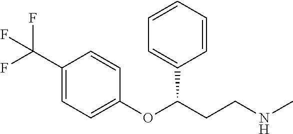 [SMILES: CNCC[C@H](Oc1ccc(C(F)(F)F)cc1)c1ccccc1] |
| R-(−)-2,5- Dimethoxy-4- bromoamphetamine hydrochloride | −2.2 | 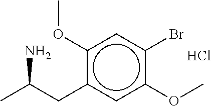 |
| | −3 | 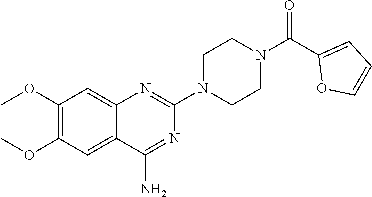 [SMILES: COc1cc2nc(N3CCN(C(=O)c4ccco4)CC3)nc(N)c2cc1OC] |
| Prazosin hydrochloride | −3 | |

### Primary Screening Assays and Counter-Screens

The triage of compounds through the pilot and primary screens is summarized in Table 4. The total number of compounds was 428,749.

TABLE 4

| | | | | | Active | Passing | Passing | |
|---|---|---|---|---|---|---|---|---|
| | | | | | (ATXN2- | Cytotox | CMV- | |
| Library | Stage | Compounds | Doses | Cells | luc) | Triage | luc | Picks |
| Multiple | MSSR | 65,728 | 1 | H2 | 330 | 12 | 4 | 2 |
| | Pilot | | | | | | | |
| LOPAC | NCATS | 1,280 | 5 | S2 | 58, 11 | 11 | 11 | n/a |
| | Pilot | | | | | | | |
| Multiple | NCATS | 361,839 | Multiple¹ | S2 | 1439 | 757 | 416 | 108 |
| | Primary | | | | | | | |

The doses were a 10 M concentration. The 330 active compounds were 0.51% of compounds screened. These compounds lowered ATXN2-luc≥2.7 SD in H2 cells. The 12 compounds passing cytotox triage were verified to lower ATXN2-luc≥2.7 SD in H2 & S1 cells, and viability by not more than 65%, at either 1 or 10 M. The 4 compounds passing CMV-luc had less than 30% reduction of CMV-luc in HC cells at 10 μM. 58 LOPAC compounds had a curve class ≤−1.1. 11 LOPAC compounds had a curve class −1.1 or −1.2. The 11 LOPAC compounds passing cytotox triage were 0.39% of compounds screened. These 11 compounds had no cytotoxicity.

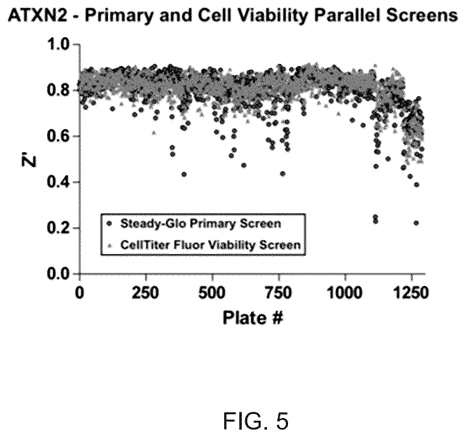

The primary screen assayed 363,021 compounds in a 1536-well screen using the primary ATXN2-luc S2 cell line assay (libraries described above). A fluorescence-based viability assay using GF-AFC substrate was used to determine compound effect on cell viability and Steady-Glo was used to measure luciferase in a multiplexed assay. The Z′-scores for the luciferase assays and the multiplexed viability assays for the largest run of 1289 1536-well plates averaged 0.8. The Z′-score vs. plate number are shown in FIG. 5.

The primary assay of 357,287 from MLSMR showed 2,763 compounds active against ATXN2-luc in S2 cells and of these 2,145 had stock solution available for follow up testing. The 2,145 compounds were evaluated in confirmation assays and tested at 5 concentrations (90 nM-57 uM), showing 1,439 compounds confirmed as active against ATXN2-luc in S2 cells, 757 were non-cytotoxic, and 416 were inactive against CMV-luc. The NCATS Pharmaceutical Collection (drug repurposing library) of 2,552 compounds was also evaluated using a similar process. This screen identified 237 compounds as active in reducing ATXN2-luc levels. Of these 237 compounds, 34 passed the viability counterscreen.

A library of 1,912 mechanistically annotated small molecules was also screened, which identified 503 compounds as active in reducing ATXN2-luc levels. This library is enriched in oncology focused compounds (reference), so a large number of the 503 compounds were also identified as cytotoxic in the viability counterscreen (202, 40%). Of the 301 remaining non-toxic compounds, 185 were also inactive in the CMV-luc counterscreen. These compounds included several molecular classes and mechanisms of action, including Hsp90 inhibitors (7 unique compounds), topoisomerase inhibitors (3 compounds) and checkpoint targeting compounds (11 compounds). Following all of the screening efforts, 46 compounds prioritized for further testing, including the Hsp9 inhibitors and glycosides which appeared as hits from several libraries.

### Secondary Assays

These 46 compounds were retested at 12 doses and a zero-compound dose, in multiplexed assays in 96-well plates using CellTiter-Fluor to determine cell viability, then Bright-Glo to determine luciferase expression. The values for H2 and S2 were used as replicates to calculate IC50s and to assess cell viability. Table 4S summarizes the screen data for the 46 compounds.

TABLE 4S

| | | Mean | min | min | min |
|---|---|---|---|---|---|
| | | IC50 | ATXN2-luc | SC luc | viability |
| Compound | Function | (μM) | (%) | (%) | (%) |
| Proscillaridin A | NaK-ATPase | 0.017 | 7.985896424 | 121.9755119 | 74.17994229 |
| | inhibitor | | | | |
| Ganetespib | HSP90 | 0.03 | 17.73512928 | 55.4123437 | 77.81518557 |
| | inhibitor | | | | |
| Camptothecin | Topoisomerase | 0.04 | 14.37151419 | 42.70840361 | 92.11146462 |
| | inhibitor | | | | |
| Luminespib | HSP90 | 0.044 | 17.88311716 | 63.25424863 | 94.63148793 |
| (NVP- | inhibitor | | | | |
| AUY922) | | | | | |
| Leptomycin B | Antibiotic, | 0.048 | 28.33295246 | 90.06213467 | 65.42422894 |
| | multifunctional | | | | |
| SNX-5422 | HSP90 | 0.088 | 19.97215451 | 69.78425811 | 87.14604608 |
| | inhibitor | | | | |
| Ouabain | NaK-ATPase | 0.122 | 6.981770302 | 122.2185702 | 74.34142278 |
| | inhibitor | | | | |
| Digoxin | NaK-ATPase | 0.129 | 9.041412146 | 107.5790491 | 67.86204468 |
| | inhibitor | | | | |
| AT7519 | CDK inhibitor | 0.162 | 14.54068563 | 103.6680368 | 50.32934722 |
| Floxuridine | pyrimidine | 0.166 | 25.65954835 | 37.50611098 | 97.92184836 |
| | analog | | | | |
| SNX-2112 | HSP90 | 0.177 | 15.51036435 | 82.28187586 | 83.30211966 |
| | inhibitor | | | | |
| Onalespib (AT- | HSP90 | 0.178 | 22.31111656 | 71.18941302 | 90.61853807 |
| 13387AU) | inhibitor | | | | |
| CHIR-124 | CHK1 | 0.219 | 17.97665977 | 69.39777453 | 75.05522951 |
| | inhibitor | | | | |
| Torin2 | mTOR | 0.257 | 25.41829914 | 128.374575 | 47.76572899 |
| | inhibitor | | | | |
| CNF-2024 | HSP90 | 0.329 | 17.8511698 | 68.16681536 | 91.84812317 |
| (BIIB021) | inhibitor | | | | |
| MK-1775 | Wee1 inhibitor | 0.343 | 21.73440059 | 63.04466782 | 77.92644315 |
| 891494-64-7 | Chk1 inhibitor | 0.388 | 25.4940694 | 91.51367341 | 87.04309783 |
| AZD-7762 | CHK1/2 | 0.416 | 21.02690518 | 70.35013155 | 78.1121656 |
| | inhibitor | | | | |
| Milciclib | CDK1 | 0.436 | 15.87587418 | 96.2245859 | 74.40455584 |
| | inhibitor | | | | |
| Tanespimycin | HSP90 | 0.442 | 23.74553195 | 96.22865559 | 83.61414369 |
| | inhibitor | | | | |
| Vosaroxin | Topoisomerase | 0.488 | 15.10445333 | 64.01021659 | 71.6055711 |
| | inhibitor | | | | |
| TCS-2312 | CK1 inhibitor | 0.545 | 20.26225528 | 54.31117653 | 61.72594215 |
| Retaspimycin | HSP90 | 0.546 | 24.20686034 | 94.99470705 | 91.49141049 |
| (IPI-504) | inhibitor | | | | |
| KW-2478 | HSP90 | 0.587 | 23.9371525 | 105.8880926 | 97.67313376 |
| | inhibitor | | | | |
| Marizomib | proteasome | 1.114 | 16.50355057 | 96.88036939 | 75.95731636 |
| | inhibitor | | | | |
| SR-3306 | JNK inhibitor | 1.298 | 15.6492787 | 82.90056981 | 65.3661759 |
| LY-2874455 | FGFR | 1.387 | 15.25622944 | 64.27199327 | 66.12261383 |
| | inhibitor | | | | |
| VER-82576 | HSP90 | 1.789 | 22.64281044 | 85.45405704 | 89.93601613 |
| (NVP-BEP800) | inhibitor | | | | |
| Amonafide | Topoisomerase | 1.836 | 21.72310257 | 69.65608767 | 68.18880407 |
| | inhibitor | | | | |
| PF-431396 | Dual | 2.318 | 15.5051748 | 98.30154577 | 64.59457936 |
| | FAK/PYK2 | | | | |
| | inhibitor | | | | |
| Brefeldin A | Autophagy | 2.89 | 44.7226174 | 84.38101604 | 72.98163788 |
| | inhibitor | | | | |
| Amuvatinib | RTK inhibitor | 3.015 | 55.06367214 | 74.25770106 | 87.65359529 |
| Roscovitine | CDK inhibitor | 3.652 | 57.32241752 | 82.446947 | 77.79096641 |
| Rebastinib | ABL1 | 3.732 | 27.07130283 | 60.58026079 | 56.5799293 |
| (DCC-2036) | inhibitor | | | | |
| BMS-345541 | IKK inhibitor | 4.054 | 20.55890524 | 96.01670561 | 77.56731696 |
| PP121 | PDGFR | 4.164 | 27.47591884 | 78.09685322 | 50.38775335 |
| | inhibitor | | | | |
| MLS000706289 | unknown | 6.848 | 61.64911256 | 79.30122089 | 89.11290891 |
| MLS000420216 | unknown | 8.083 | 56.46853156 | 72.15852365 | 84.51618025 |
| BX-912 | PDK1 | 8.887 | 54.93947796 | 100.3512435 | 84.39704756 |
| | inhibitor | | | | |
| GW843682X | PLK kinase | 9.772 | 57.1311382 | 87.9923365 | 75.49792925 |
| | inhibitor | | | | |
| Silmitasertib | CK2 inhibitor | 10.18 | 45.64161707 | 93.78008929 | 77.63154447 |
| MLS000035284 | unknown | 12.862 | 38.56578802 | 4.093821213 | 76.84592989 |
| Gandotinib | Jak2 inhibitor | 21.116 | 23.15027157 | 67.18574352 | 81.63551151 |
| Melphalan | DNA | 24.119 | 44.83723881 | 79.96642533 | 89.25488874 |
| | alkylating | | | | |
| | agent | | | | |
| PRT-060318 | Syk inhibitor | 25.4 | 64.25347993 | 86.82043789 | 94.61970724 |
| GSK-615 | PI3Kalpha | 57.473 | 51.44189205 | 85.55137127 | 75.52699002 |
| | inhibitor | | | | |

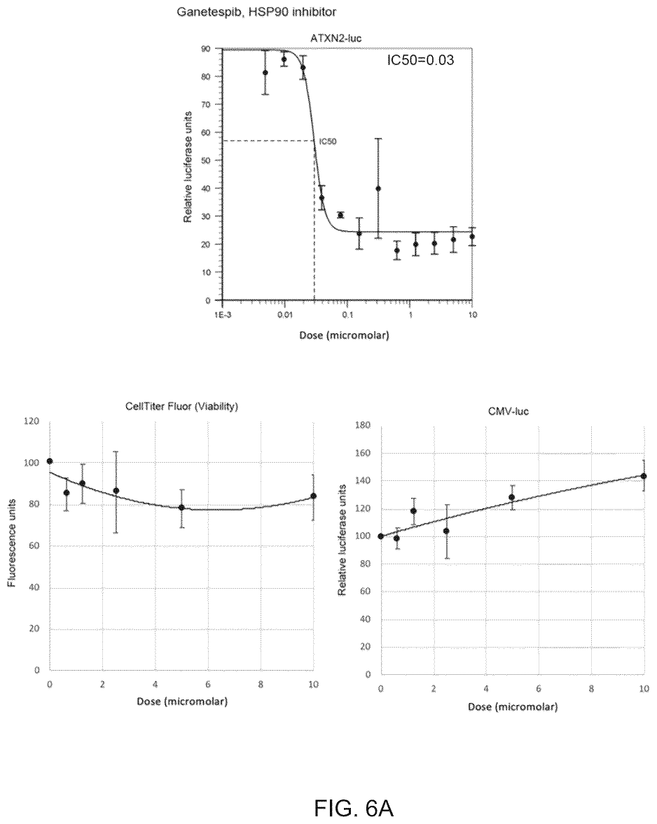

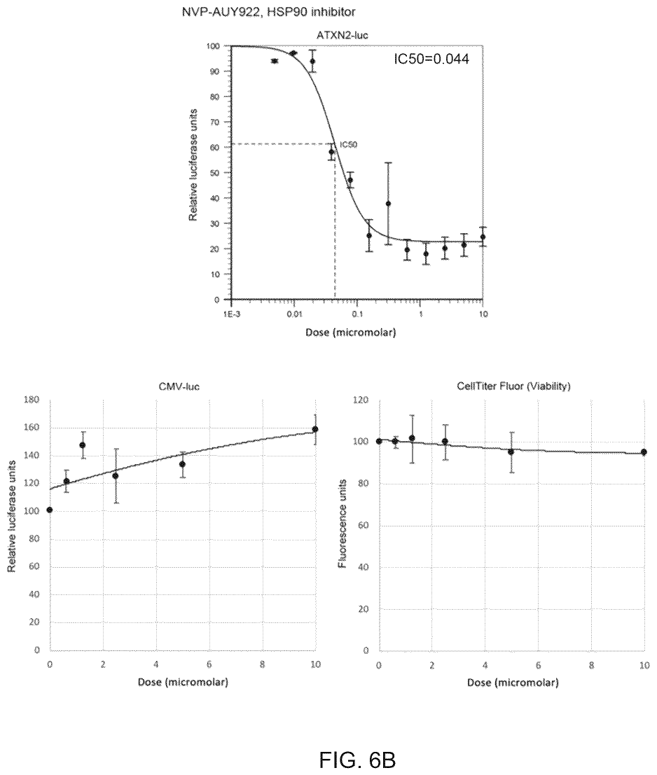

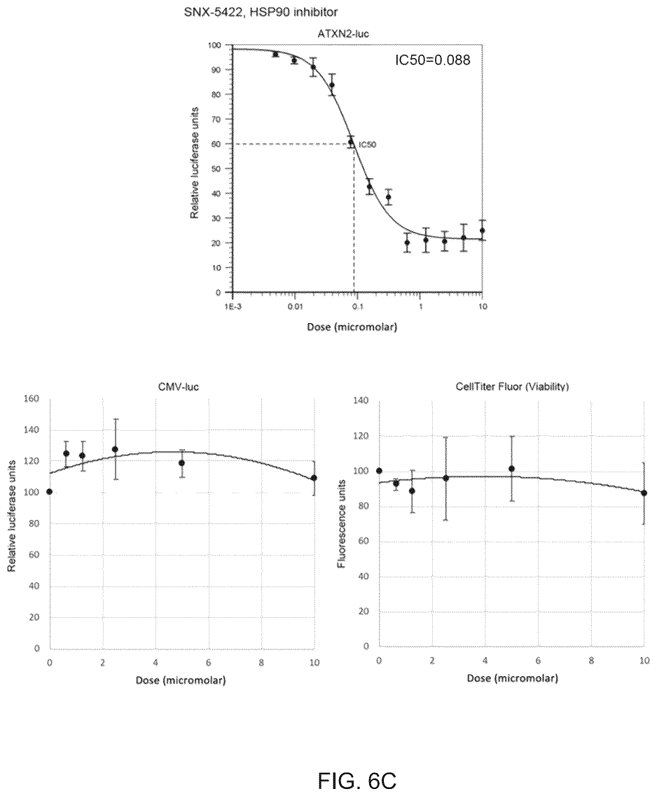

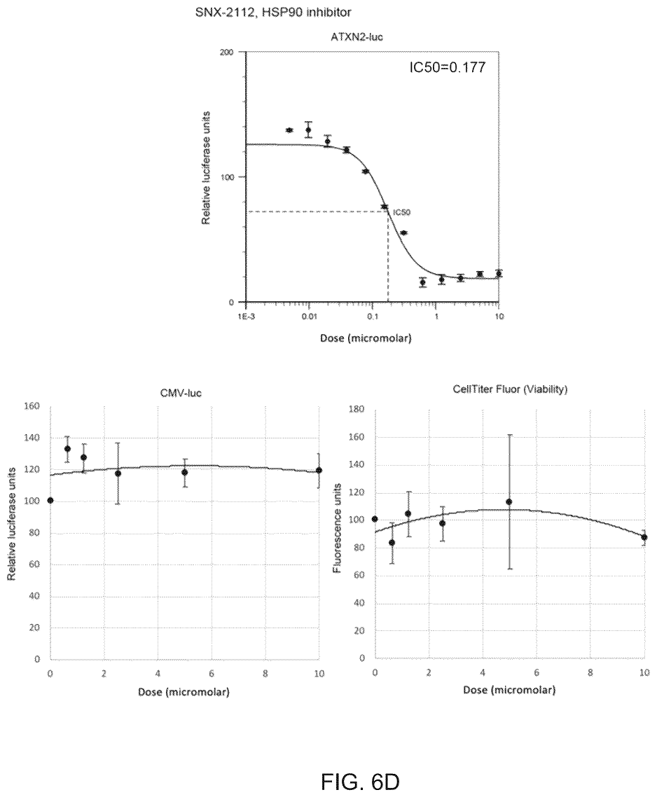

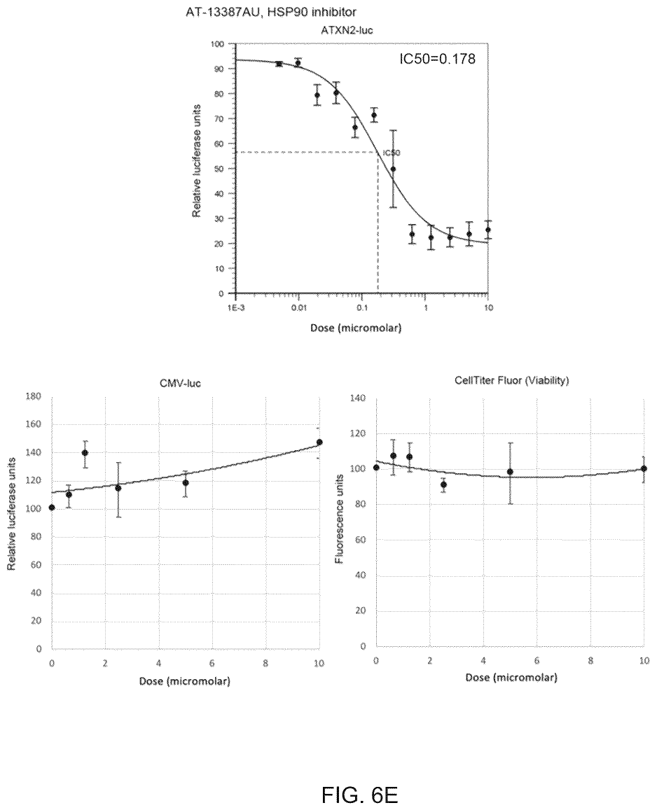

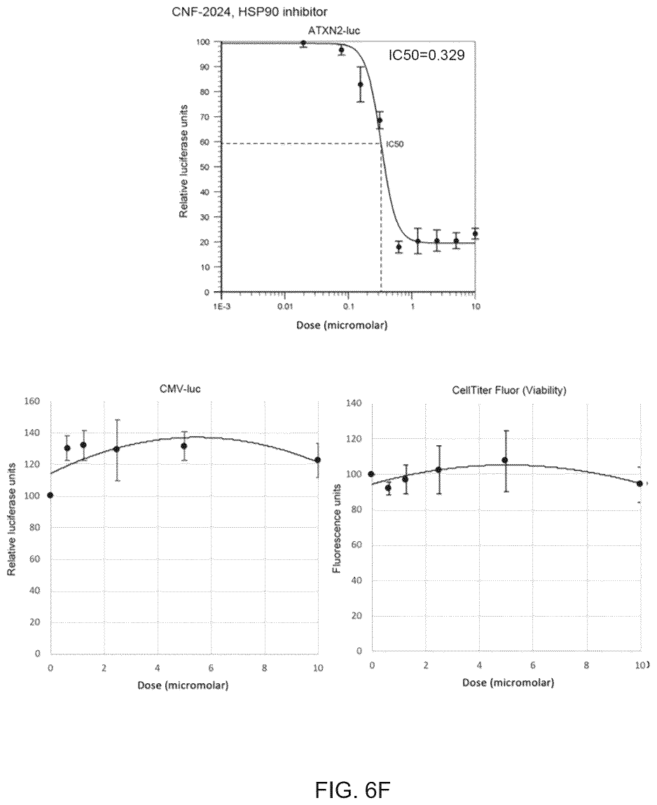

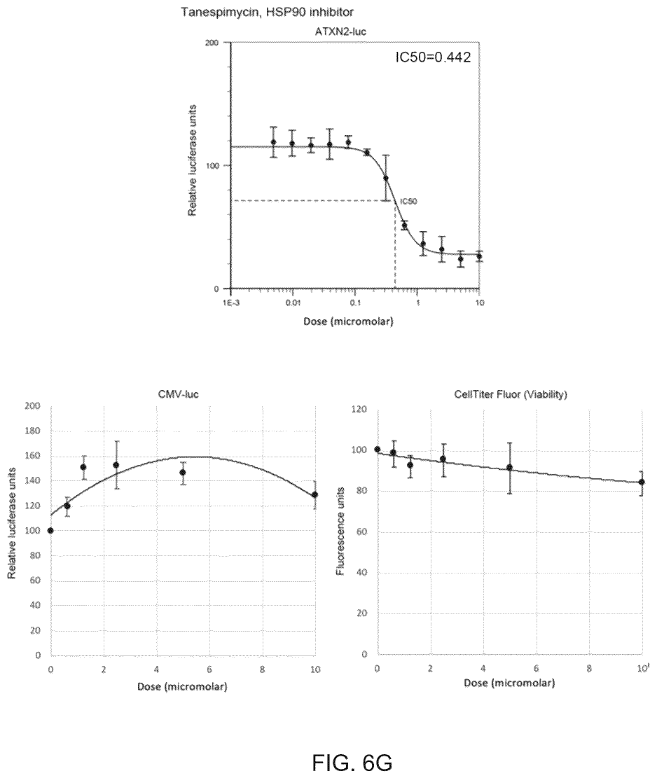

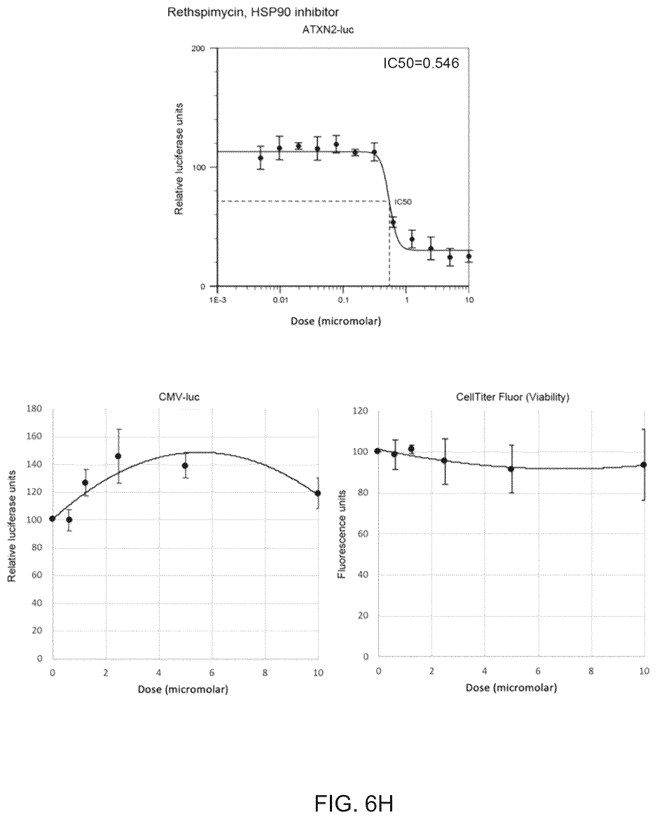

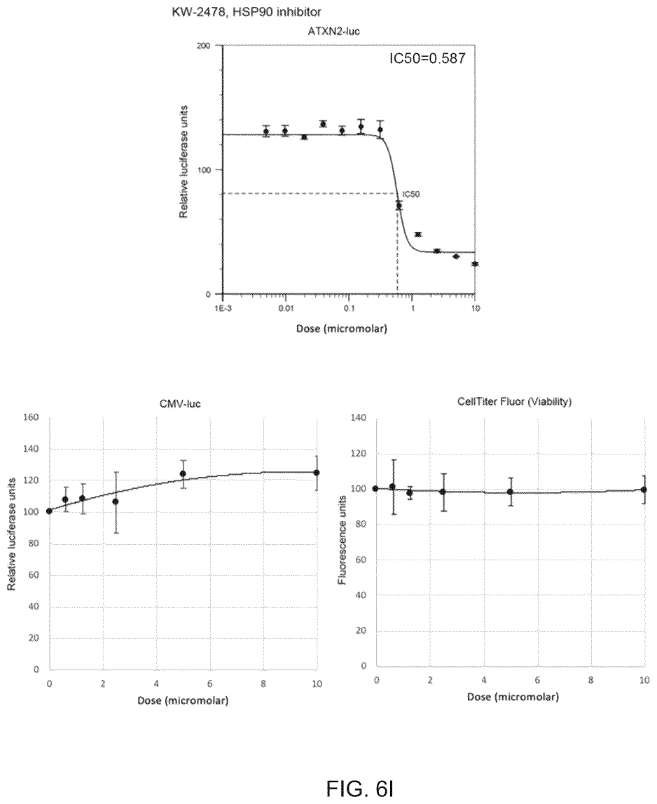

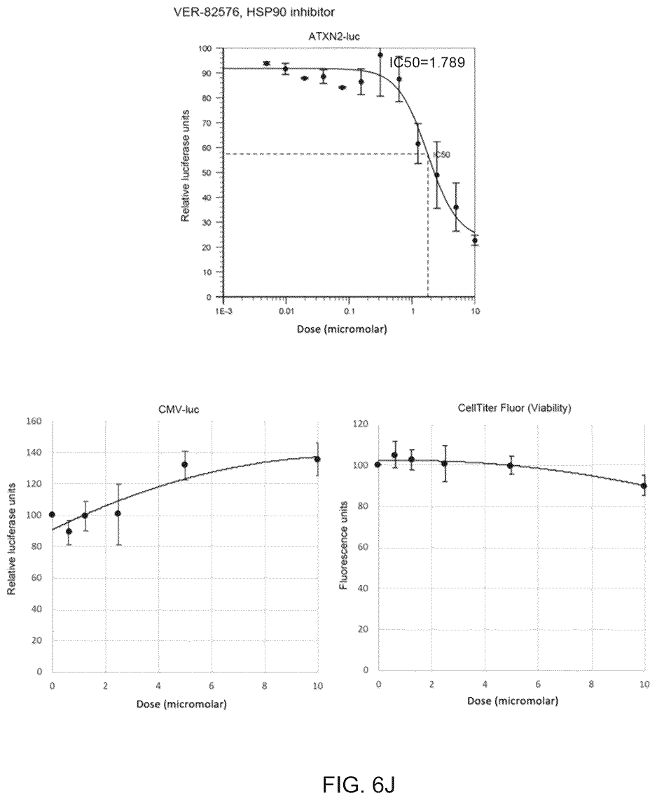

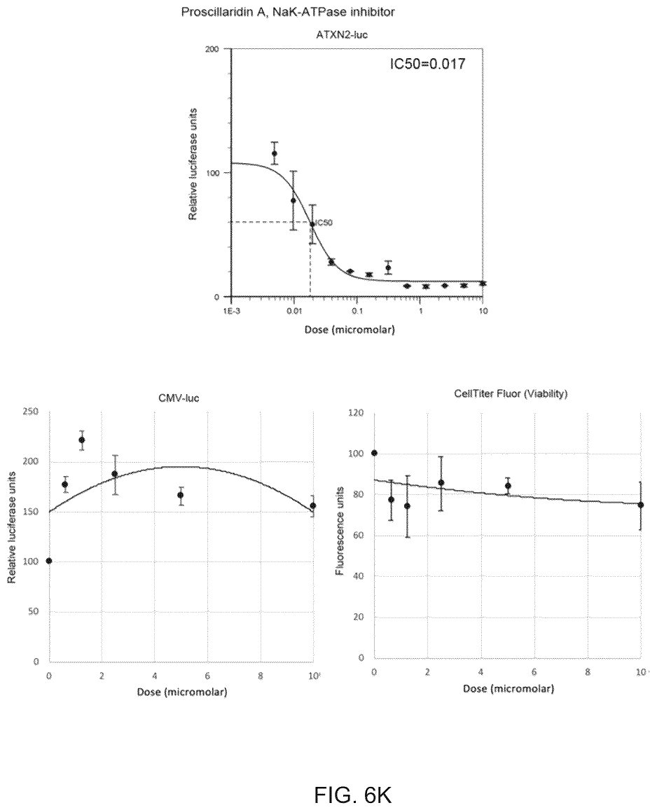

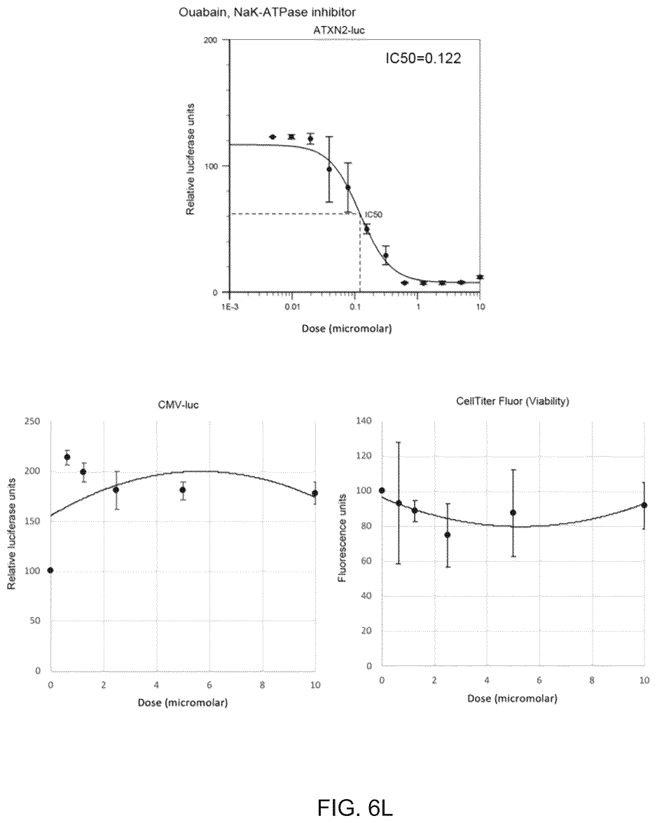

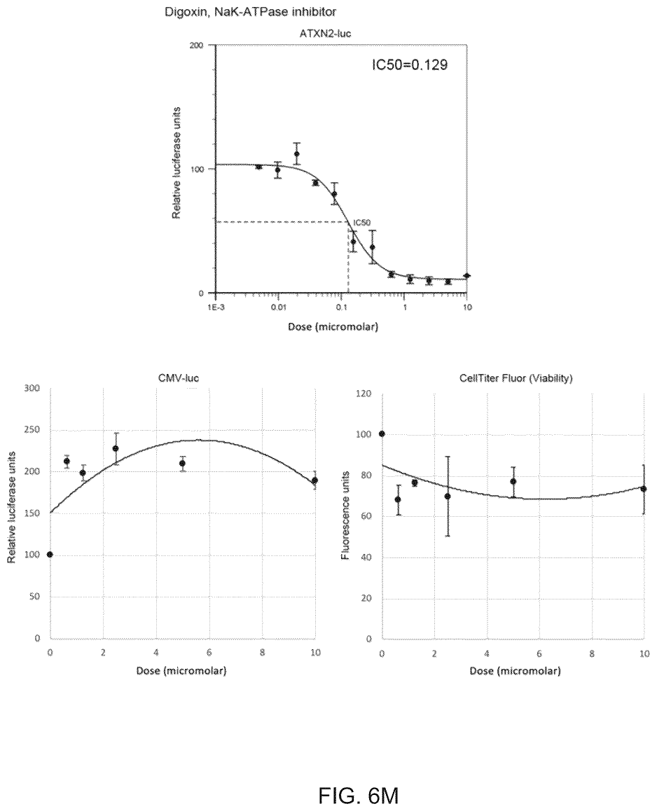

Among these compounds were 10 HSP90 inhibitors, 3 NaK-ATPase inhibitors (cardiac glycosides), 3 topoisomerase inhibitors, 7 compounds targeting checkpoint signaling (CDK, CHK, WEE1), and 2 casein kinase (CK) inhibitors. No other known targeted functions were common among the remaining 21 compounds. Among all of the 46 compounds, the two with the lowest IC50s were the NaK-ATPase inhibitor proscillaridin A (17 nM) and the HSP90 inhibitor Ganetespib (30 nM). Graphs comparing ATXN2-luc, CMV-luc both with separate viability assessments for all of the HSP90 inhibitors and NaK-ATPase inhibitors are provided in FIGS. 6A-6M. These figures show results for the following HSP90 inhibitors and NaK-ATPase, in order: ganetespib, NVP-AUY922, SNX-5422, SNX-2112, AT-13387AU, CNF-2024, tanespimycin, rethspimycin, KW-2478, VER-82576, proscillaridin A, ouabain, and digoxin. For each compound, a first graph shows relative luciferase units (RLUs) were averaged for H2 and S2 cells expressing ATXN2-luc (n=2 cell lines). The fitted line was determined using the Hill coefficient method for determining IC50s, shown on the charts. A second graph shows RLUs for SC cells expressing CMV-luc (n=1 cell line). A third graph shows average viability for H2, S2, and SC cells determined using CellTitor Fluor (n=3). For CMV-luc and viability charts, the fitted lines are second-order polynomials. Means and SD are shown.

### Orthogonal Assays

For selected compounds targeting NaK-ATPase and HSP90, the ability of compounds to also reduce endogenous ATXN2 expression in HEK-293 cells was verified:

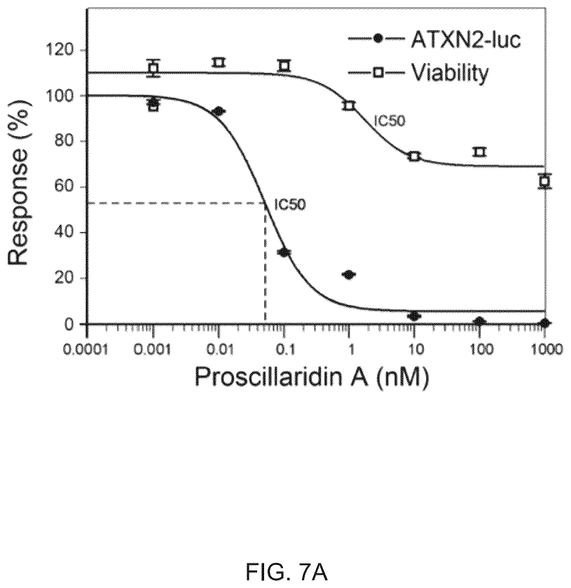

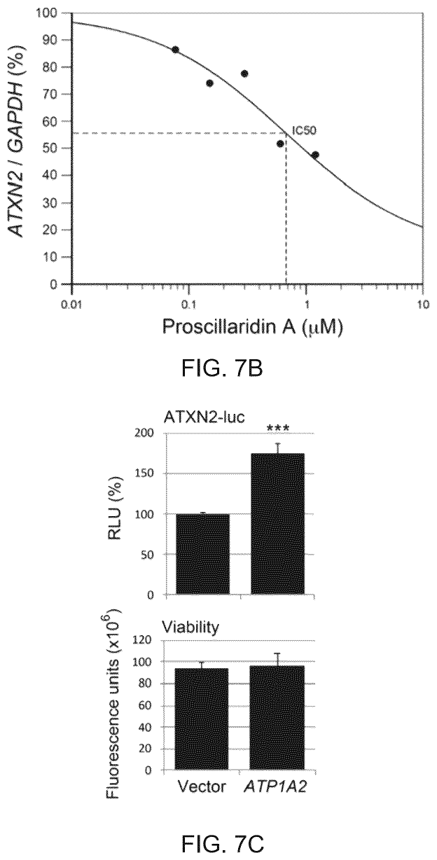

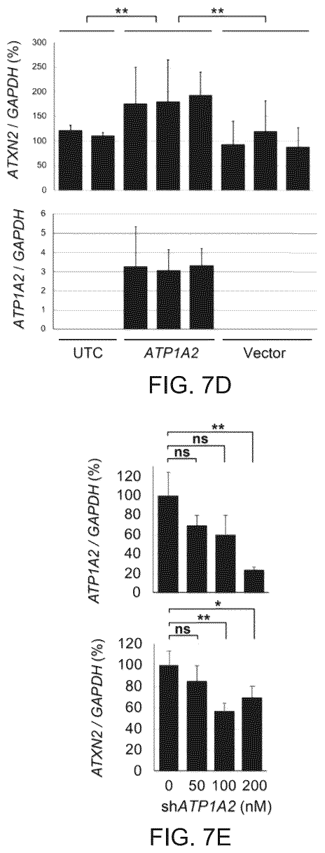

Proscillaridin A: Proscillaridin A had the lowest IC50 of any compound tested in the secondary assays, at 17 nM. Dose-dependent reduction of ATXN2-luc was reconfirmed in H2 cells with paired viability assays using the MTT method, confirming ATXN2-luc reduction by 80% at 1 nM with a minimal effect on cellular viability. ATXN2 expression associates with the expression of the proscillaridin A target ATP1A2. FIG. 7A shows ATXN2-luc expression in H2 cells treated for 48 hrs with the indicated doses of proscillaridin A. The cells were evaluated for ATXN2-luc expression by luciferase assay. The IC50 was 0.052 nM. The cells were also evaluated for viability by MTT assay. Values shown are means and standard deviations. HEK-293 cells were then treated with increasing doses of proscillaridin A for either 24 and 48 hours, showing >50% reduction of endogenous ATXN2 when cells were treated with 1 nM proscillaridin A at 48 hours. FIG. 7B shows the endogenous ATXN2 in the HEK-293 cells after 48 hrs of proscillaridin A treatment vs 24 hrs, determined by qPCR. The IC50 was 0.677 μM.

Cardiac glycosides such as proscillaridin A generally target three proteins, ATP1A1, ATP1A2 and ATP1A3. Of these ATP1A2 is expressed highly in Purkinje cells (Allen Brain Atlas). When ATP1A2 was overexpressed in H2 cells followed by evaluating ATXN2-luc expression by luciferase assay, the expression of ATXN2-luc was doubled. FIG. 7C shows ATXN2-luc expression (top) of S2 cells that were transfected with ATP1A2, and the cell viability (bottom) determined by CellTiter-Fluor assay. Similarly, when ATP1A2 was overexpressed in HEK-293 cells and evaluated ATXN2 expression by quantitative real time-PCR (qPCR), the expression of ATXN2 was significantly increased. FIG. 7D shows means and standard deviations from independent transfections, including the cells transfected with ATP1A2, each analyzed by qPCR. Finally, the expression of ATP1A2 was reduced in HEK-293 cells by RNA interference (RNAi) resulting in reduced endogenous ATXN2 transcription, as determined by qPCR. FIG. 7E shows the ATP1A2 and ATXN2 transcription means and standard deviations. Statistical tests were Student's t-test (C) or ANOVA and post-hoc Bonferroni corrected t-tests (D, E). *, p<0.05; **, p<0.01; ***, p<0.001; ns, not significant.

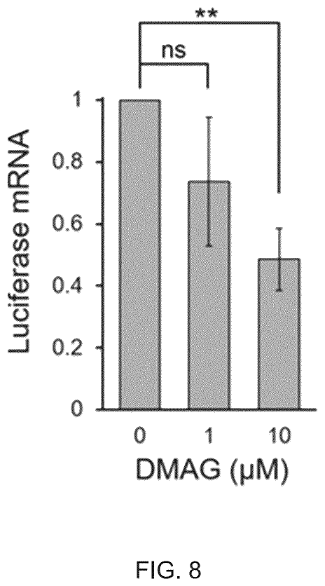

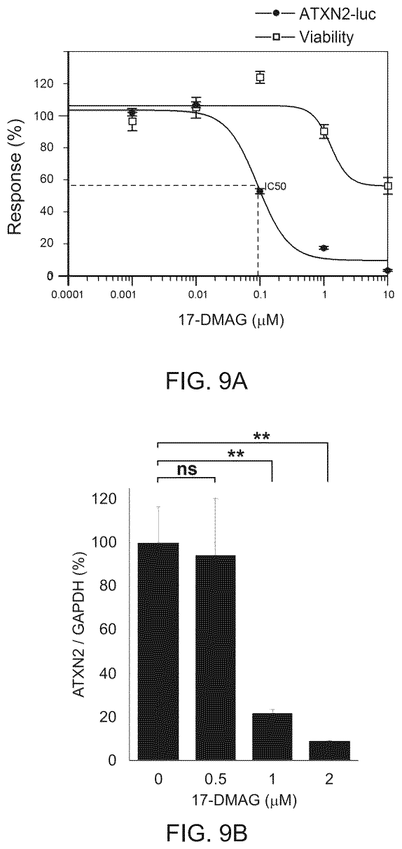

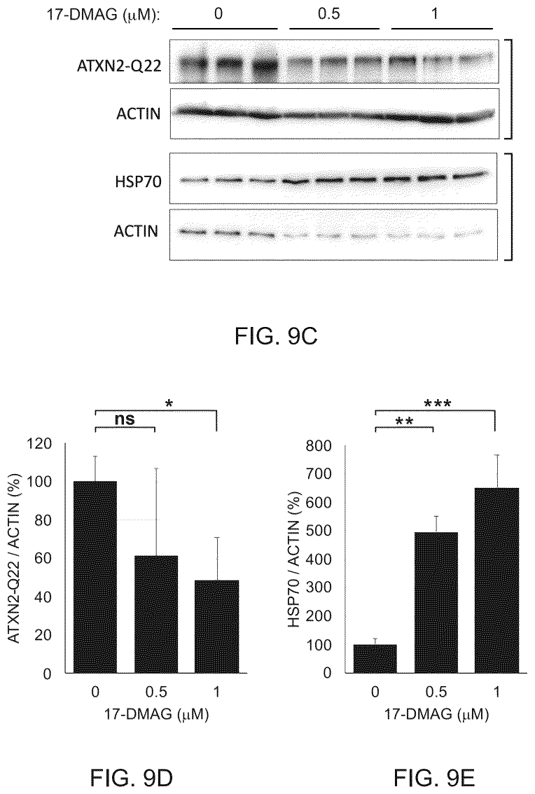

HSP90 inhibitors: Ten HSP90 inhibitors were identified in the primary screen: ganetespib, luminespib (NVP-AUY922), SNX-5422, SNX-2112, onalespib (AT-13387AU), CNF-2024 (BIIB021), tanespimycin, retaspimycin, KW-2478 and VER-82576 (NVP-BEP800). One additional HSP90 inhibitor was also identified in the validation screen (derrubone). This supports HSP90 inhibitors as very effective for lowering ATXN2 expression; with ganetespib (IC50=30 nM) exhibiting the second most potent activity for lowering ATXN2-luc expression. To characterize a bioavailable HSP90 inhibitor, 17-dimethylaminoethylamino-17-demethoxygeldanamycin (17-DMAG, also known as alvespimycin) was characterized for inhibition of ATXN2 in cultured cells. First, it was verified that 17-DMAG treatment of H2 cells resulted in the reduction of ATXN2-luc transcription in H2 cells treated with 1 and 10 uM 17-DMAG for 48 hrs. FIG. 8 shows the luciferase mRNA vs. concentration of 17-DMAG that was used to treat H2 cells. The ATXN2-luc mRNA abundance was determined by quantitative PCR using primers that amplify the luciferase gene. Probabilities are from one-way ANOVA and post-hoc Bonferroni corrected t-tests: ns, not significant; **, P<0.01. Next, H2 cells were treated with increasing doses of 17-DMAG followed by luciferase assays, which revealed an IC50 for lowering ATXN2-luc of 93 nM (FIG. 9A). 17-DMAG also reduced the expression of endogenous ATXN2 transcription in HEK-293 cells in a dose-dependent manner, determined by qPCR, with an IC50 of 759 nM (FIG. 9B). This pattern parallels that for proscillaridin A, where the IC50 for lowering endogenous ATXN2 transcription in HEK-293 cells that was also an order of magnitude greater than that for lowering ATXN2-luc in the H2 reporter cells, again suggesting the result might be due to a higher ATXN2-luc copy number vs endogenous ATXN2 (see above). 17-DMAG also reduced the expression of endogenous non-mutant ATXN2 protein (FIG. 9C) and mutant ATXN2-Q58 protein in HEK-293 KI cells (FIG. 9D), determined by western blotting. Successful inhibition of HSP90 can be demonstrated by upregulation of HSP70, as HSP90 inhibitors block HSP90 interaction with the HSF1 transcription factor, which then forms homotrimers that translocate to the nucleus and transactivate HSP70 and other HSP genes (FIG. 9E).

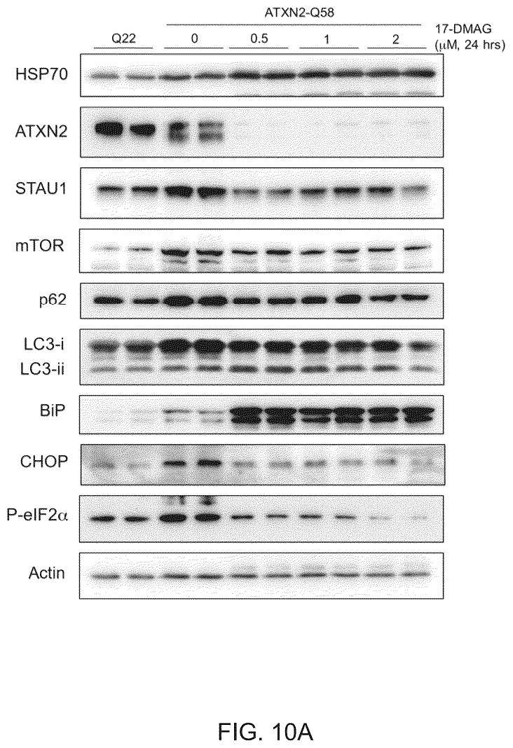

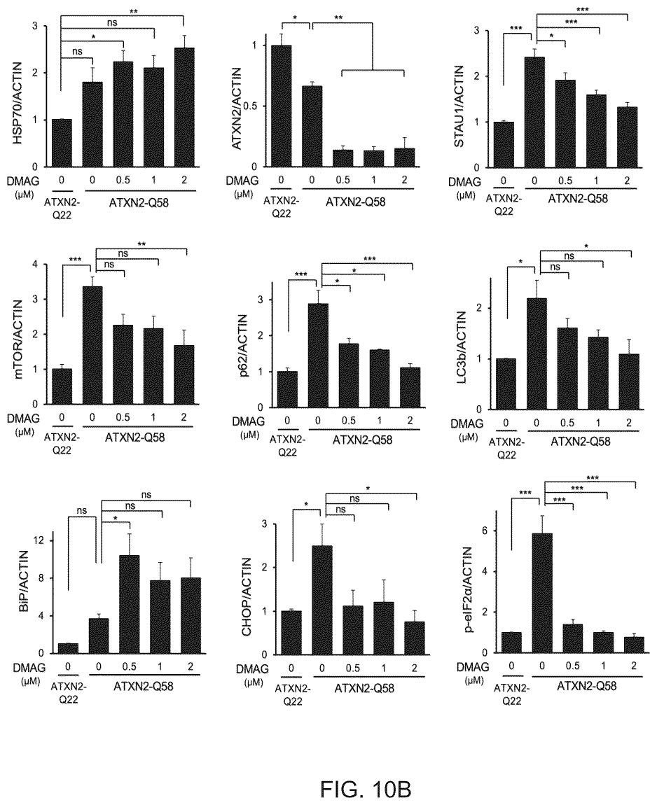

To determine if lowering ATXN2-Q58 in HEK-293 KI cells could restore pathways that are abnormal in SCA2, several proteins were evaluated by western blotting and RNAs by quantitative PCR following 17-DMAG treatment. Again, elevated HSP70 was observed with 17-DMAG treatment, consistent with HSP90 target engagement. When ATXN2-Q58 in HEK-293 KI cells were treated with 17-DMAG, significantly reduced mutant ATXN2 protein was observed, along with restored abundance of STAU1 and autophagy marker proteins (mTOR, p62, LC3b) and proteins functioning in the unfolded protein response (UPR) including CHOP and eIF3α, and further elevated BiP. FIG. 10A shows these proteins in non-mutated cells (Q22) and mutated cells (ATXN2-Q58) at multiple concentrations of 17-DMAG. FIG. 10B shows means and standard deviations of these proteins in the non-mutated cells and mutated cells at multiple concentrations of 17-DMAG. Probabilities are from one-way ANOVA and post-hoc Bonferroni corrected t-tests: ns, not significant; *, p<0.05; **, p<0.01, ***, p<0.001. The n number of blots ranged from 3 to 9.

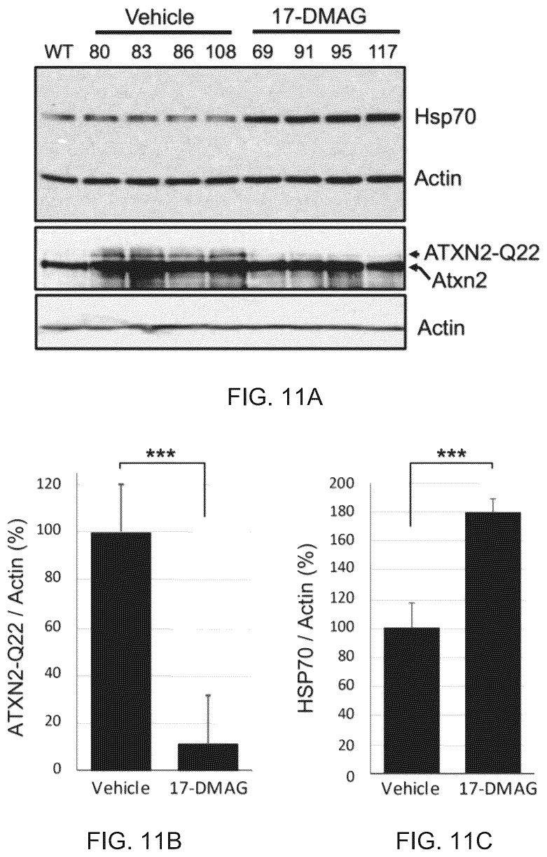

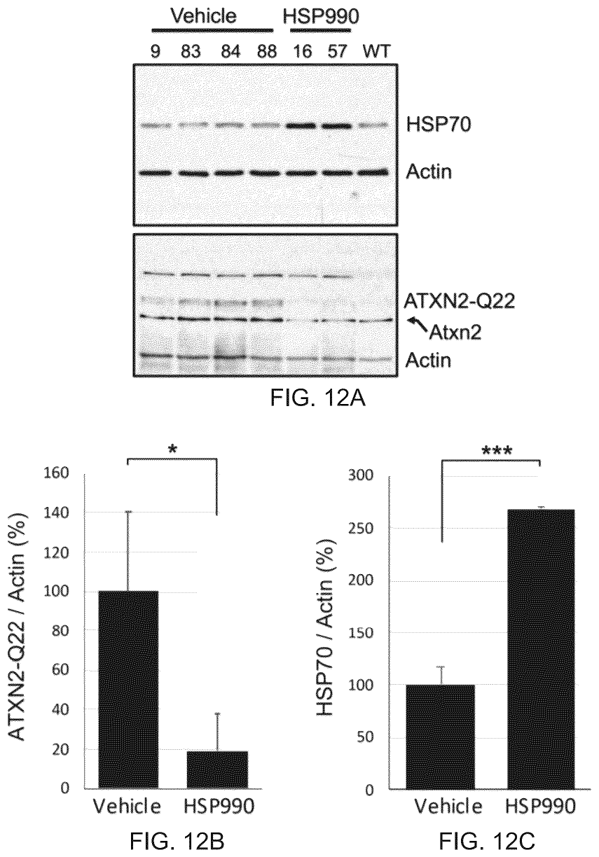

Being a bioavailable compound able to cross the blood brain barrier, 17-DMAG was tested to determine whether it inhibited ATXN2 expression in a bacterial artificial chromosome (BAC) ATXN2-Q22 transgenic mouse model, described previously. Since 17-DMAG reduced expression of both the wildtype and mutant ATXN2 proteins, its effects were tested using only ATXN2-BAC mice harboring the non-mutant human ATXN2-Q22 gene (BAC-Q22 mice). Reliable detection of mutant ATXN2 protein by western blot in samples isolated from SCA2 BAC mice is possible, but it is challenging and involves use of the 1C2 antibody. To test efficacy of 17-DMAG in the wildtype ATXN2 mice, 27 wk old BAC-Q22 mice were treated (n=4 per group, 2 males, 2 females) every other day over 16-days (days 1, 3, 5, 7, 9, 11, 13, 15) then sacrificed on day 16. The expression of ATXN2 and HSP70 were evaluated by western blots, which showed 17DMAG treatment reduced ATXN2 abundance, which was associated with HSP70 induction. FIG. 11A shows the proteins for each mouse, with the mouse ID numbers above the lanes. FIG. 11B shows the means and standard deviations of ATXN2-Q22 in mice treated with the vehicle and with 17-DMAG. FIG. 11C shows the means and standard deviations of HSP70 in mice treated with the vehicle and with 17-DMAG. A similar experiment was performed using a second HSP90 inhibitor of a distinct chemotype that can also cross the blood brain barrier, HSP990. For HSP990, 25 wk old BAC-Q22 mice were treated (n=4 per group, 2 males, 2 females) every other day over 10 days (days 1, 3, 5, 7 and 9) then sacrificed on day 10. Unexpectedly, the two females in the HSP990 treatment group did not survive. The expression of ATXN2 and HSP70 were evaluated on western blots, and like for 17-DMAG mice, HSP990 treatment also reduced ATXN2 abundance and increased HSP70, for the surviving mice. FIG. 12A shows the proteins for each mouse, with the mouse ID numbers above the lanes. FIG. 12B shows means and standard deviations of ATXN2-Q22 in mice treated with the vehicle and with HSP990. FIG. 12C shows the means and standard deviations of HSP70 in mice treated with the vehicle and with HSP990. Probabilities are from Student's t-tests: *, p<0.05; ***, p<0.001. Three blots were quantified.

### Discussion

Over the past decade ATXN2 has emerged as a potential therapeutic target for not only SCA2 but also for ALS and perhaps other neurodegenerative disorders. CAG repeat expansion mutation in ATXN2 results in a toxic gain of function for the encoded ATXN2 protein. Lowering ATXN2 expression improved phenotypes in SCA2 and ALS mice. A goal of the screening described herein was to identify ATXN2-lowering compounds that can serve as scaffolds in the development of small molecule therapeutics for SCA2 and potentially for ALS.

### Targeting ATXN2 in SCA2 and ALS

ATXN2 has been indicated as a therapeutic target for both SCA2 and ALS. In SCA2 mice, lowering ATXN2 expression restored the expression of cerebellar proteins and mRNAs, restored Purkinje cell firing frequency, and improved the SCA2 motor rotarod phenotype. Targeting ATXN2 may also be an effective therapeutic approach for treating ALS. Sequencing of ATXN2 in ALS patients showed that intermediate CAG repeat expansions in ATXN2 increased ALS risk. Hypothetically then, lowering ATXN2 abundance might be an effective approach to treating ALS. This hypothesis was tested by crossing a TDP-43 mice with Atxn2 knockout mice, and by lowering Atxn2 expression in TDP-43 mice using an ASO, demonstrating improved survival and reduced numbers of RNA-granules positive for TDP-43 or ATXN2 proteins.

Additionally, ATXN2 mutation results in abnormal signaling in pathways regulating autophagy and the unfolded protein response (UPR). In cultured SCA2 patient fibroblast cell lines and in cerebellar and spinal cord tissues of SCA2 mouse models, hyperactivated mTOR signaling was observed, resulting in autophagy inhibition marked by increased LC3-II. This was attributed to increased mTOR mRNA translation mediated by direct mRNA interaction by the stress-related protein Staufen1 (STAU1) which was also highly elevated in these SCA2 cells and models. Likewise, in HEK293 cells expressing mutant ATXN2-Q58, mutant PERK/CHOP signaling was observed, indicating UPR activation. It has been found that STAU1, mTOR-related autophagy proteins, and UPR proteins CHOP and phospho-eIF2α are restored in ATXN2-Q58 HEK293 cells by 17-DMAG treatment. Interestingly, a significant increase in BiP was found in response to 17-DMAG. BiP is a key ER chaperone of the HSP70 family that functions as the ER paralog of HSP90. As such, BiP elevation might be compensatory to HSP90 inhibition, but might be highly context dependent. In ATXN2-Q58 cells, which display basal pro-apoptotic UPR activation and autophagy dysfunction, the decrease in pro-apoptotic CHOP associated with a BiP increase could mark a switch to a restorative and adaptive activation of the UPR.

STAU1 abundance and autophagy readouts could be restored by RNAi targeting either STAU1 or ATXN2, and UPR pathways could be restored by RNAi targeting STAU1. Moreover, elevated STAU1 and hyperactive mTOR signaling with elevated LC3-II were observed in TDP-43 ALS patient fibroblasts and spinal cords of TDP-43 mice, that was restored when mice were haplo-insufficient for STAU1. Collectively, these data support that lowering ATXN2 expression can restore abnormal STAU1, autophagy and UPR signaling associated with disease in SCA2 and ALS.

Lowering ATXAN2 Expression with Cardiac Glycosides

Many cardiac glycosides were found in the pilot screen. Among the compounds selected following the primary screen there were three, including proscillaridin A, ouabain and digoxin, of which proscillaridin A had the lowest IC50. Cardiac glycosides are inhibitors of sodium potassium ATPases (NaK ATPases) and are used to treat congestive heart failure and cardiac arrythmia. Cardiac glycosides are particularly toxic and able to trigger apoptosis, yet can be antiapoptotic, promoting cellular growth at low doses. Overexpression of the Purkinje cell abundant NaK ATPase ATP1A2 in HEK-293 cells increased ATXN2 expression, while lowering ATP1A2 expression by RNAi, reduced ATXN2 transcription.

Collectively, these data demonstrate that proscillaridin A and likely other cardiac glycosides regulate ATXN2 abundance transcriptionally, at least in part via regulating ATP1A2. More work would be needed to demonstrate cardiac glycosides lower ATXN2 abundance in vivo, as efforts to accomplish this in a similar way as for 17-DMAG in SCA2 mice were unsuccessful.

ATXN2 and one of its interactors, STAU1, colocalize with TDP-43 and SG proteins.

Lowering ATXN2 expression using cardiac glycosides may modify RNA-granules. A hallmark feature of SCA2 is cytoplasmic inclusion bodies in Purkinje cells. The stress granule protein STAU1 becomes highly (up to 6-fold) elevated in SCA2 and ALS models.

As described above, reduction of expression of the non-mutant ATXN2 gene in TDP-43 models reduced the abundance of ATXN2 and TDP-43 positive stress granules. Additionally, ATXN2 mutation was associated with increased numbers of TIA-1 positive stress granules in HEK-293 cells and in SCA2 patient fibroblasts. In SCA2 mice haplo-insufficient for Stau1, the SCA2 cerebellar molecular phenotype was nearly restored, the motor phenotype was improved, and Purkinje cell inclusion bodies were reduced in numbers. A study by Fang et al. identified cardiac glycosides as modulating SG phenotypes, but did not elucidate the molecular mechanism or action via the canonical target of cardiac glycoside action. The data suggest that SG modulation is mediated—at least in part—through reduction of ATXN2 and the ATP1A2 pump.

Lowering ATXN2 Expression with HSP90 Inhibitors

This study identified 11 compounds known to inhibit HSP90, including one that was identified in the original pilot screen. Although not identified in the screens, 17-DMAG (alvespimycin) was examined because of its prior use in human clinical trials. Furthermore, it is orally bioavailable and able to cross the blood brain barrier. 17-DMAG inhibited transcription of ATXN2 in HEK-293 cells in a dose dependent manner, and reduced the abundance of endogenous and mutant ATXN2 in HEK-293 cells. Remarkably, ATXN2-BAC mice treated by 17-DMAG had 90% reduction of ATXN2 protein abundance. Another bioavailable HSP90 inhibitor, HSP990, was also used in a clinical trial but was associated with dose-limiting neurotoxicities. Toxicity was also evident in the in vivo experiment described herein. While mice well-tolerated 17-DMAG, half of the mice treated with HSP990 did not survive treatment. However, like for 17-DMAG, ATXN2-BAC mice treated with HSP990 had 80% reduced ATXN2 expression.

Evidence of the possible molecular action of 17-DMAG on ATXN2 expression came from another spinocerebellar ataxia study, on SCA3. ATXN3-135Q transgenic mice treated with 17-DMAG had reduced ATXN3 protein abundance in CNS tissues and improved motor coordination, but ATXN3 mRNA was only moderately reduced. ATXN3 inhibition by 17-DMAG was associated with reduced ATXN3 aggregates in the brainstem and evidence for modified autophagic flux. 17-DMAG was also effective for clearing aggregates of huntingin protein in mammalian cells, and for improving polyglutamine aggregates and motor phenotypes in a spinal and bulbar muscular atrophy (SBMA) mouse model. These studies raise hope for SCA2, in which HSP90 inhibitors may act dually, by inhibiting ATXN2 transcription while also modifying autophagic flux to reduce ATXN2 protein abundance and pathogenic aggregates.

In conclusion, following a qHTS screen of 428,749 compounds, multiple compounds were identified falling into three classes (cardiac glycosides, HSP90 inhibitors, and topoisomerase inhibitors) that lower or otherwise modulate ATXN2 expression, as well as lead compounds acting in other pathways. The identification of these compounds advances understanding on mechanisms involved in ATXN2 expression and protein and aggregate turnover. Compounds selected from the lead list may serve as therapeutics for SCA2 or scaffolds for optimizing SCA2 therapeutics by medicinal chemistry.

Table 5S lists 43 annotated compounds grouped by their function, with the number of standard deviations below the mean that was found in the pilot screen.

TABLE 5S

| | | SD from |
|---|---|---|
| Compound | Function if Known | Mean |
| Emetine dihydrochloride | calcium channel blocker | −4.840 |
| TMB-8 | Calcium channel blocker | −5.740 |
| SKF-96365 | Calcium channel blocker | −2.721 |
| NICARDIPINE | Calcium channel blocker | −2.782 |
| U-50488 | Calcium channel blocker | −2.834 |
| Digitoxigenin | cardiac glycoside | −3.629 |
| Digoxin | cardiac glycoside | −3.904 |
| GITOXIGENIN | cardiac glycoside | −4.261 |
| DIACETATE | | |
| CYMARIN | cardiac glycoside | −4.099 |
| STROPHANTHIDINIC | cardiac glycoside | −3.855 |
| ACID LACTONE | | |
| ACETATE | | |
| SARMENTOGENIN | cardiac glycoside | −3.539 |
| DIGITOXIN | cardiac glycoside | −4.100 |
| Helveticoside | cardiac glycoside | −2.733 |
| STROPHANTHIDIN | cardiac glycoside | −4.451 |
| EMICYMARIN | cardiac glycoside | −3.080 |
| PERUVOSIDE | cardiac glycoside | −4.038 |
| OUABAIN | cardiac glycoside | −3.832 |
| CONVALLATOXIN | cardiac glycoside | −3.689 |
| Digoxigenin | cardiac glycoside | −3.264 |
| Lanatoside C | cardiac glycoside | −3.629 |
| Proscillaridin A | cardiac glycoside | −7.684 |
| PERIPLOCYMARIN | cardiac glycoside | −2.759 |
| LANATOSIDE C | cardiac glycoside | −4.323 |
| GITOXIN | cardiac glycoside | −4.086 |
| NERIIFOLIN | cardiac glycoside | −3.045 |
| SU1498 | Flk-1 kinase inhibitor | −4.885 |
| Sanguinarine | inhibits glutamate | −2.689 |
| | decarboxylase | |
| Cephaeline dihydrochloride | maybe serotonin and | −4.055 |
| heptahydrate | dopamine receptors | |
| Cyanocobalamin | one of the B-12 vitamins | −2.681 |
| U-37883A | potassium channels | −4.965 |
| PINACIDIL | potassium channels | −4.875 |
| Trifluridine | pyrimidine nucleoside | −5.158 |
| | antiviral | |
| Menadione | vitamin K3 | −3.434 |
| TRIFLUOPERAZINE | antipsychotic | −5.856 |
| HYDROCHLORIDE | | |
| RESORCINOL | antiseborrheic antipruritic | −6.242 |
| MONOACETATE | | |
| Indatraline | blocks reuptake of dopamine | −3.071 |
| | norepinephrine serotonin | |
| LOMERIZINE DiHCl | calcium channel blocker | −2.782 |
| L-694 247 | dopamine receptor agonist | −3.156 |
| DERRUBONE | HSP90 inhibitor | −3.621 |
| TEGASEROD MALEATE | serotonin 5-HT4 agonist | −3.606 |
| 5-Nonyloxytryptamine | serotonin receptor agonist | −3.607 |
| Tramadol | u-opioid receptor | −3.415 |
| | noradrenergic serotonergic | |
| COBALAMINE | Vitamin B12 | −5.111 |

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements may be devised without departing from the spirit and scope of the described technology.

The foregoing detailed description describes the disclosure with reference to specific exemplary embodiments. However, it will be appreciated that various modifications and changes can be made without departing from the scope of the present disclosure as set forth in the appended claims. The detailed description and accompanying drawings are to be regarded as merely illustrative, rather than as restrictive, and all such modifications or changes, if any, are intended to fall within the scope of the present disclosure as described and set forth herein.

1. A method of modulating ATXN2 expression in a cell comprising administering to the cell an effective amount of an ATXN2 modulating agent selected from the group consisting of a cardiac glycoside, an HSP90 inhibitor, an NaK-ATPase inhibitor, a topoisomerase inhibitor, or a combination thereof.

2-3. (canceled)

4. The method of claim 1, wherein the ATXN2 modulating agent is a cardiac glycoside.

5. The method of claim 4, wherein the cardiac glycoside is selected from the group consisting of strophantin octahydrate, dihydroouabain, lanatoside B, helveticoside, ouabain, and combinations thereof.

6. The method of claim 4, wherein the cardiac glycoside excludes proscillaridin A, digoxin, digitoxigenin, and strophanthidin.

7. The method of claim 1, wherein the ATXN2 modulating agent is an HSP90 inhibitor.

8. The method of claim 7, wherein the HSP90 inhibitor is selected from the group consisting of HSP990, Ganetespib, Luminespib, SNX-5422, SNX-2112, Onalespib, CNF-2024, Tanespimycin, Retaspimycin, KW-2478, VER-82576, and combinations thereof.

9. The method of claim 7, wherein the HSP90 inhibitor excludes 17-DMAG.

10. The method of claim 1, wherein the ATXN2 modulating agent is an NaK-ATPase inhibitor.

11. The method of claim 10, wherein the NaK-ATPase inhibitor is selected from the group consisting of strophantin octahydrate, dihydroouabain, lanatoside B, helveticoside, ouabain, and combinations thereof.

12. The method of claim 10, wherein the NaK-ATPase inhibitor excludes proscillaridin A, digoxin, digitoxigenin, and strophanthidin.

13. The method of claim 1, wherein the ATXN2 modulating agent is a topoisomerase inhibitor.

14. The method of claim 13, wherein the topoisomerase inhibitor is selected from the group consisting of camptothecin, vosaroxin, amonafide, etoposide, mitoxantrone dihydrochloride, and combinations thereof.

15. The method of claim 1, wherein the cell has a mutated ATXN2 gene.

16. The method of claim 15, wherein the mutated ATXN2 gene includes a tract of repeating CAG codons having 33 or more consecutive CAG codons.

17. The method of claim 1, wherein the ATXN2 modulating agent inhibits ATXN2 expression with an IC50 of from about 17 nM to about 57 μM.

18. The method of claim 1, wherein administering to the cell an effective amount of an ATXN2 modulating agent comprises exposing the cell to the ATXN2 modulating agent at a concentration from about 10 nM to about 10 μM.

19. The method of claim 1, wherein the ATXN2 expression is reduced by greater than about 50%.

20. The method of claim 1 wherein a cellular protein concentration of ataxin-2 is reduced by greater than 50%.

21. The method of claim 1, wherein the ataxin-2 includes a polyglutamine expansion from a mutated ATXN2 gene.

22. The method of claim 1, wherein the ATXN2 modulating agent excludes proscillaridin A, digoxin, digitoxigenin, strophanthidin, and 17-DMAG.

23-25. (canceled)


SEQUENCE LISTING (14 sequences)

SEQ ID NO: 1 - DNA, 29 bp, synthetic construct
  source 1..29: mol_type = other DNA
cctcggtcgacagcccaagcttggcactg

SEQ ID NO: 2 - DNA, 29 bp, synthetic construct
  source 1..29: mol_type = other DNA
cttggagtggtgaatccgttagcgaggtg

SEQ ID NO: 3 - DNA, 21 bp, synthetic construct
  source 1..21: mol_type = other DNA
gttgacattgattattgacta

SEQ ID NO: 4 - DNA, 30 bp, synthetic construct
  source 1..30: mol_type = other DNA
cgctaccggtttattgttcatttttgagaa

SEQ ID NO: 5 - DNA, 21 bp, synthetic construct
  source 1..21: mol_type = other DNA
gttgacattgattattgacta

SEQ ID NO: 6 - DNA, 18 bp, synthetic construct
  source 1..18: mol_type = other DNA
gagctctgcttatataga

SEQ ID NO: 7 - DNA, 25 bp, synthetic construct
  source 1..25: mol_type = other DNA
aagatatggactccagttatgcaaa

SEQ ID NO: 8 - DNA, 20 bp, synthetic construct
  source 1..20: mol_type = other DNA
caaagcctcaagttcctcat

SEQ ID NO: 9 - DNA, 24 bp, synthetic construct
  source 1..24: mol_type = other DNA
aagacccgccgcaactcagtcttc

SEQ ID NO: 10 - DNA, 24 bp, synthetic construct
  source 1..24: mol_type = other DNA
accaggtgactttgagcgggtaca

SEQ ID NO: 11 - DNA, 21 bp, synthetic construct
  source 1..21: mol_type = other DNA
gaagatggtgatgggatttcc

SEQ ID NO: 12 - DNA, 21 bp, synthetic construct
  source 1..21: mol_type = other DNA
gaaggtgaaggtcggagtcaa

SEQ ID NO: 13 - DNA, 22 bp, synthetic construct
  source 1..22: mol_type = other DNA
cgtcgacaacggctccggcatg

SEQ ID NO: 14 - DNA, 23 bp, synthetic construct
  source 1..23: mol_type = other DNA
gggcctcgtcacccacataggag